(19) United States
(12) Patent Application Publication
(10) Pub. No.: US 2023/0002817 A1
(43) Pub. Date: Jan. 5, 2023

(54) METHOD AND KIT FOR DETECTING GENOME EDITING AND APPLICATION THEREOF

(71) Applicant: SHANDONG UNIVERSITY, Qingdao, Shandong (CN)

(72) Inventors: Qilai HUANG, Qingdao (CN); Bo LI, Qingdao (CN)

(73) Assignee: SHANDONG UNIVERSITY, Qingdao, Shandong (CN)

(21) Appl. No.: 17/619,140

(22) PCT Filed: Jun. 12, 2020
(86) PCT No.: PCT/CN2020/095927

§ 371 Date: Dec. 14, 2021

(30) Foreign Application Priority Data
Jun. 14, 2019 (CN) 201910516755.X

(51) Int. Cl. C12Q 1/6851; C12Q 1/6858

(52) U.S. Cl. CPC C12Q 1/6851; C12Q 1/6858; C12Q 2521/101; C12Q 2531/113; C12Q 2545/113

(57) ABSTRACT

A method and a kit for detecting genome editing and application thereof belongs to the field of genome editing efficiency detection, and the getPCR method for determining genome editing efficiency includes quantifying wild-type DNA in a genome to be tested and calculating the percentage of the wild-type DNA to determine the genome editing efficiency. The method has been proved to have good detection accuracy and simple operation, and can be applied to all genome editing methods to quantify genome editing efficiency and screen single-cell clones.

### TECHNICAL FIELD

The present application belongs to the field of genome editing efficiency detection, and specifically relates to a method for indirectly confirming the probability of genome editing by determining the proportion of wild-type genomic DNA, and its application in the evaluation of genome editing efficiency and monoclonal screening.

### BACKGROUND

The information disclosed in the background of the present application is intended to enhance an understanding of the general background of the present application and should not necessarily be taken as an acknowledgement or any form of suggestion that this information has become known prior art to those of ordinary skill in the art.

CRISPR/Cas9 is a major genome editing technology and is widely used, and its gene modification effect is associated with small guide RNA (sgRNA). In the CRISPR/Cas9 system, Cas9 nuclease is directed to target DNA containing the protospacer adjacent motif (PAM) by single guide RNA (sgRNA), then cleaves both strands of target DNA at a site 3 bp upstream of the PAM sequence and generates double-strand breaks (DSBs) Once sensed, the DSBs will be repaired mostly by two different kinds of intrinsic mechanisms, homology-directed repair (HDR) or non-homologous end joining (NHEJ). NHEJ involves direct ligation of broken ends without the need for a homologous template and repairs DNA breaks in an error-prone manner. The NHEJ usually leads to unpredictable insertion or deletion of bases at DNA breaks in the genome, named indels. This strategy can be applied to gene knockout and have been widely used in gene function studies and in clinical to remove pathogenic genes.

In CRISPR-Cas9-mediated genome editing, pre-screening of excellent sgRNAs is important to obtain good editing efficiency and specificity, and efficient sgRNAs are preferred to obtain single-cell clones or offsprings with desired alterations. The current widely used methods to evaluate the genome editing efficiency are mainly based on DNA sequencing or mismatch-specific nucleases. Sanger sequencing method involves PCR amplification and cloning steps of the target region before each DNA sequence being read separately. This multistep method can provide detailed information of each mutation event induced by nuclease, but is quite time-consuming, costly and laborious. The next-generation DNA sequencing (NGS) technology was also applied in profiling DNA mutation induced by sgRNA-directed Cas9 nuclease owing to its massive parallel capacity. Several web-based online platforms have been developed to analyze the NGS data, including CRISPR-GA, BATCH-GE, CRISPResso, Cas-analyzer and CRISPRMatch et al. However, even though effective, these NGS-based methods still require multi-step operations and are costly in time and money. The mismatch specific nuclease-based approach is currently the most popular method that employs T7 endonuclease 1 (T7E1) or Surveyor nuclease to cleave double-stranded DNA containing mismatched bases formed between DNA strands containing sequence differences between the two DNA strands that are caused by genome editing, allowing for the detection of editing efficiency. This method has the advantage of requiring only basic laboratory devices, but is not applicable to the detection of single nucleotide polymorphic regions and often misses single nucleotide mutations as well as large fragment deletions. In addition, scientists have developed many other alternatives, but only improved in some aspects, such as qEva-CRISPR21, engineered nuclease-induced translocation (ENIT), Cas9 nuclease-based restriction fragment length polymorphism (RFLP) analysis, Indel detection by amplicon analysis (IDAA), and gene editing frequency digital PCR (GEF-dPCR). The inventors consider that the experimental steps of the above technologies are cumbersome, and the PCR amplification products of the target DNA regions rather than directly using genomic DNA itself are used to quantify editing efficiency. It is widely known that sequence- and length-dependent biases introduced during PCR amplification will inevitably affect the accuracy of the assay.

### SUMMARY

In view of the above research background, the inventors believe that it is of great significance to provide a method which is supposed to be simple in experiment procedure, reliable in quantification result, time-saving and low cost as well as not requiring specific devices that not readily available in major laboratories. The present application provides a method for detecting the genome editing efficiency, named genome editing test PCR (getPCR). The getPCR utilizes the selective amplification characteristic of Taq polymerase in amplifying the wild-type DNA in the genome DNA to be tested to determine the proportion of the wild-type DNA by quantitatively amplifying the wild-type DNA in the amplification product, and further judges the occurrence frequency of indel in the genome to be tested. The detection result is more accurate and has wide application potential. The method has good accuracy when applied to indel detection induced by endonuclease Cas9 and can be applied to the detection of genome editing efficiency related to Cas9 nuclease technology, such as the evaluation of sgRNA performance, HDR efficiency, and base editor in the CRISPR/Cas9 system; besides, it can also be used for the confirmation and screening of single-cell clone genotypes.

The following technical solutions are provided in this disclosure.

In a first aspect of the present application, a method for detecting the frequency of nuclease-induced indel occurrence is provided, wherein the method comprising: adding primers and Taq DNA polymerase to a genomic DNA sample to be detected, amplifying wild-type DNA in the genomic DNA sample, and quantifying the proportion of wild-type DNA by PCR, thereby confirming the frequency of indel occurrence in the genome; the primers are sequence-matched to the wild-type DNA and the sequence of the primers cover the cutting site of the nuclease.

Preferably, the nucleases include, but are not limited to, Cas9 nucleases, zinc finger nucleases (ZFNs), transcription activator-like effector nucleases (TALENs), CRISPR RNA guide FokI nucleases (RFNs), and paired cas9 nickases. Further, the nucleases are Cas9 nucleases.

Zinc finger nucleases (ZFNs), transcription activator-like effector nucleases (TALENS) and CRISPER-Cas9 systems are commonly used in modern genetic engineering techniques, and it is important to provide reliable and simple methods for evaluating the efficiency of these genetic modification techniques. The efficiency of CRISPR sgRNA is usually evaluated by quantifying the frequency of indel occurrence in the field, and real-time PCR technology is the most effective method in nucleic acid quantification. However, the diversity and unpredictability of indel occurrence make it impossible to design indel-specific primers, so technicians cannot directly quantify indel frequency by real-time PCR. The method described in the first aspect, namely getPCR technology, selectively amplifies wild-type DNA in the genome and quantifies the proportion of wild-type DNA by the relative quantification strategy of real-time PCR to bypass this barrier. Taq polymerase is able to specifically amplify templates that exactly match the primer without amplifying templates that mismatch with the primer and Taq polymerase has a low tolerance to base mismatches between primers and complementary sequences. The herein disclosed method utilizes selective amplification by Taq polymerase, which allows accurate quantification of wild-type DNA and thus obtains the probability of occurrence of indel. In some embodiments of the present application, good detection was achieved by primer design and optimization of primer parameters for nuclease cutting sites with targeted cleavage function, using Cas9 nuclease as an example. It is demonstrated that the research ideas and technical solutions of the present application are feasible and expected to have good results as a detection method for a variety of gene editing technologies.

Preferably, in some embodiments of the present invention, the PCR quantification is real-time PCR or ddPCR.

It is further preferred that the amplification reaction (or amplifying mentioned in some embodiments) refers to performing real-time PCR, wherein the annealing temperature of the amplification reaction is Tₘ˜Tₘ4° C.

Preferably, the detection method further comprises the step of introducing a control amplification at a position hundreds of base pairs away from the cutting site and calculating the percentage of wild-type DNA in the edited genomic DNA sample by AACt strategy.

Preferably, the primer is designed to span Cas9 nuclease cutting site near its 3′ end.

Preferably, the primer comprises a watching sequence, the watching sequence is a sequence between the nuclease cutting site and the 3′ end of the primer, having a length of 1 to 8 bp.

Further preferably, the primer is a nucleotide sequence, and the length of the watching primer is 3 to 5 bp.

Further preferably, the primers is a pair of nucleotide sequences designed in forward and reverse direction, and the length of the watching primer base is 4 bp.

Further preferably, the 3′ end base of watching primer is an adenine base or a cytosine or a guanine base; more preferably, the 3′ end base of watching primer is an adenine base.

In a second aspect of the present application, a kit for detecting the frequency of nuclease-induced indel occurrence is provided, the kit comprising primers, Taq DNA polymerase and PCR detection reagents; the use of the kit can perform the detection method as described in the first aspect.

In a third aspect of the present application, applications of the kit described in the second aspect in evaluating genome editing efficiency, and/or single-cell clone screening are provided.

Preferably, the genome editing comprising NHEJ mediated indels, HDR-mediated genome modification and base editing generated by BE4.

Preferably, the application of the kit further comprising the screening of gRNAs adapted for CRISPR.

In a fourth aspect, a method for genotyping of single-cell clones is provided, wherein the method comprising: using wild-type DNA in genome to be tested as a template, designing primers against alleles, extracting genomic DNA of single-cell clones to be tested, and detecting whether the alleles in the genomic DNA of single-cell clones have indels by the method described in the first aspect thereby achieving single-cell colony genotyping.

In a fifth aspect of the present application, a method for detecting HDR efficiency is provided, the detection method comprising: designing primers for the genomic DNA repaired by HDR in the genome to be detected, extracting the genomic DNA to be detected, and detecting the occurrence probability of HDR by adopting the method in the first aspect; the percentage of DNA repaired by HDR is the HDR efficiency.

In a sixth aspect of the present application, a method for detecting the editing efficiency of a base editor is provided, the detection method comprising: taking the genomic DNA to be detected as a template, designing primers for a target sequence after base editing, and adopting the detection method described in the first aspect to detect the occurrence probability of base editing in the genome, which is the editing efficiency of the base editor.

In the embodiment of this application, taking the applications in Lenti-X 293 T cells on 8 sgRNA targets as examples, it is indicated that getPCR technique could determine the genome editing efficiency accurately in all cases of genome editing including NHEJ induced indels, HDR and base editing. Meanwhile, this method exhibited great power in single-cell clone genotyping by its ability in telling exactly how many alleles were modified.

Compared with the prior arts, the present application has the following beneficial effects:

1. With the rapid development and wide application of CRISPR technology, it is important to provide a simple, accurate and reliable method to evaluate the efficiency of genome editing for the screening of gRNA and the optimization of experimental protocols. The method provided by the present application is simple in experiment procedure, reliable in quantification result, time-saving and low cost as well as not requiring specific devices that not readily available in major laboratories, and requires only one qPCR step. getPCR accurately determined indel frequencies at CRISPR targets with comparable results to NGS based methods, which was believed to be the most reliable one.

2. Cas nuclease-based gene editing methods are all available using the methods of the present application, including NHEJ-induced indel, HDR and base editing, and can also be applied to the screening of single-cell clones.

3. getPCR provides a common way to evaluate the genome modifications generated by RNA guided nucleases. It can be easily further extended for use in genome editing evaluation of other nucleases which have predictable cutting position, including zinc finger nuclease, transcription activator-like effector nucleases and CRISPR RNA-guided Fokl nuclease, paired Cas9 nickases. This method will hopefully further boom the wide application of genome editing technologies in molecular and cellular biology researches in the future by further defining the design rules for watching primers.

### BRIEF DESCRIPTION OF THE DRAWINGS

The accompanying drawings, which are incorporated in and constitute a part of this application, are included to provide a further understanding of the application, and the description of the exemplary embodiments and illustrations of the application are intended to explain the application and are not intended to limit the application.

FIG. 1: Principle and flowchart of getPCR.

(a) Principle of getPCR in discriminating indel and wild sequences. (b) Overview of getPCR strategy.

FIG. 2: Principle of getPCR primer design in Example 1.

(a) Twenty-six plasmids constructed to mimic indels at HOXB13 gene gRNA target 4.

(b) Sixteen types of watching primers with different number of watching bases; evaluation of their ability in discriminating indels for reverse primers (c) and forward primers (d) and the combination of forward and reverse primers (e), respectively.

(f) Investigation of the background self-amplification signal when forward and reverse primers are used in combination.

(g) Influence of the amplification base at primer 3′ end on PCR amplification specificity.

(h) Effect of different mismatch types on PCR amplification efficiency.

(i) The role of the type of 3′ terminal base in determining the sensitivity of getPCR to mismatches. (Means±s.e.m, n=3 independent technical replicates).

FIG. 3: Parameter optimization for running getPCR in example 2.

(a-d) Amplification curves of DNA templates with or without indels using four watching primers at different annealing temperature. The watching primers contain three (a) or four (b) watching bases in forward direction or three (c) or four (d) watching bases in reverse direction respectively.

(e-h) Line charts showing the influence of watching primers Tm value on the PCR efficiency and selectivity over indels at different annealing temperature in PCR amplification, using forward watching primers with three (e) or four (g) watching bases and reverse watching primers with three (f) or four (h) watching bases. PCR efficiency is shown as ΔCt calculated relative to Ct value at 65° C. and selectivity is shown as ΔCt between wild type and indel templates. Watching primer sequences are shown in the bottom. The small circle denotes the best selectivity under optimum amplification efficiency at 0.5 cycle dropped Ct value as indicated by the dashed line.

(i-1) Influence of annealing temperature on PCR amplification efficiency and the linearity of standard curve, characterized by R square value. Four watching primers employed in the examination are forward with three (i) or four (k) watching bases and reverse with three (j) or four (1) watching bases respectively (Means±s.e.m, n=3 independent technical replicates).

FIG. 4: Application of getPCR in editing frequency determination and single-cell colony genotyping on indel-mimic plasmids.

(a) Surveyor assay electrophoresis chromatogram of a sample containing a given percentage of insertion deletions, used to simulate genomically edited DNA.

(b) Apparent editing frequencies from quantified Surveyor assay results.

(c) On the same indels mimics, indel frequencies were determined using getPCR method with forward and reverse watching primer alone or in combination.

(d-f) Genotyping of mimic single-cell clones using three differently designed getPCR watching primers. (Means ±s.e.m, n =3 independent technical replicates, *P<0.05, **P<0.01, <0.001).

FIG. 5: Result graph of the determination of editing frequency and genotype of single-cell clones by getPCR in example 5.

Indel frequency determination and single-cell colony genotyping in Lenti-X 293 T cells genomically edited by gRNA targeting on HOXB13, DYRKIA and EMX1 genes.

(a) Application of getPCR in quantification of indel frequency generated at eight gRNA in comparison with NGS and Surveyor methods.

(b) Illustration of gRNA sequences and watching primers employed in getPCR, single-cell clones isolated and propagated from edited Lenti-X 293 T cells with sgRNA targeting HOXB13 gene (c, d), EMXI gene (e, f, i) and DYRK1A gene (g, h) were genotyped by getPCR methods. Box plots show amplification quartile, median, and third quartile, with whiskers indicating 1.5 IQR, and outliers shown separately. The correlation and combination effects of two different designs of watching bases were assessed in genotyping (j-1). (Means ±s.e.m, n=3 independent technical replicates, *P<0.05, **P<0.01,<0.001).

FIG. 6: Result graph of application of getPCR in HDR detection in cells and genotype of single-cell clones in example 6.

(a) Schematic overview of the getPCR principle in detection of HDR and base editing.

(b) Demonstration of getPCR watching primers designed for evaluating HDR efficiency in EMX1 gene and base editing in EMX1 and HOXB13 genes.

(c) HDR efficiency quantification with getPCR in comparison with NGS and HindIII digestion methods.

(d-f) Single cell clones were isolated and propagated from HDR experiment and genotyped by getPCR method with two different watching primers alone or in combination. Box plots show amplification quartile, median, and third quartile, with whiskers indicating 1.5 IQR, and outliers shown separately.

(g, h) Frequency of each genotype determined by getPCR and NGS method in base editing experiment targeting EMX1 and HOXB13 gene respectively.

(i) Detailed genotypes of 10 clones from EMX1 gene base editing experiment which are heterozygous at both 5^{th} and 6^{th} position were further determined by getPCR method.

(j, k) Bar chart and scatterplots display genotyping results of 5^{th} nucleotide of EMX1 gene of single-cell clones from base editing experiment.

(1, m) Single-cell clone genotyping of the 6^{th} nucleotide of EMX1 gene in base editing experiment.

(n, o) Bar chart and scatterplots display of genotyping results of single-cell clones underwent base editing on HOXB13 gene. (Means±s.e.m, n=3 independent technical replicates, *P<0.05, **P<0.01, <0.001).

FIG. 7 Considerations in designing getPCR primers and running getPCR.

(a, b) Design of multiple getPCR primers with given watching bases but different length/Tm value, in forward and reverse direction respectively.

(c) Amplification efficiency of these getPCR primers on wild type template.

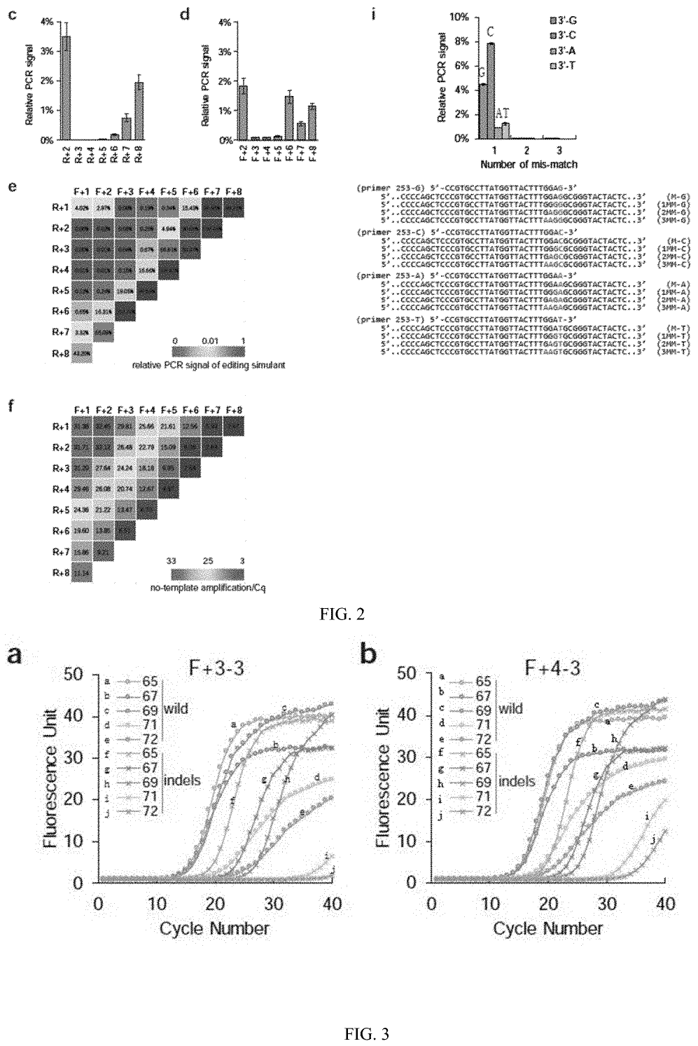

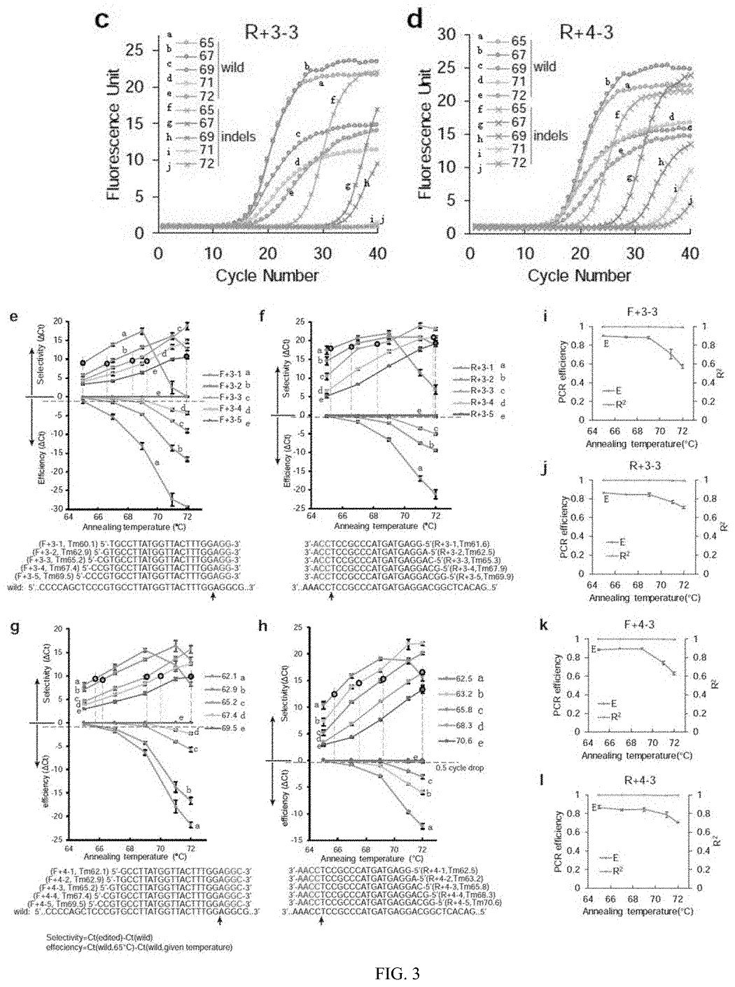

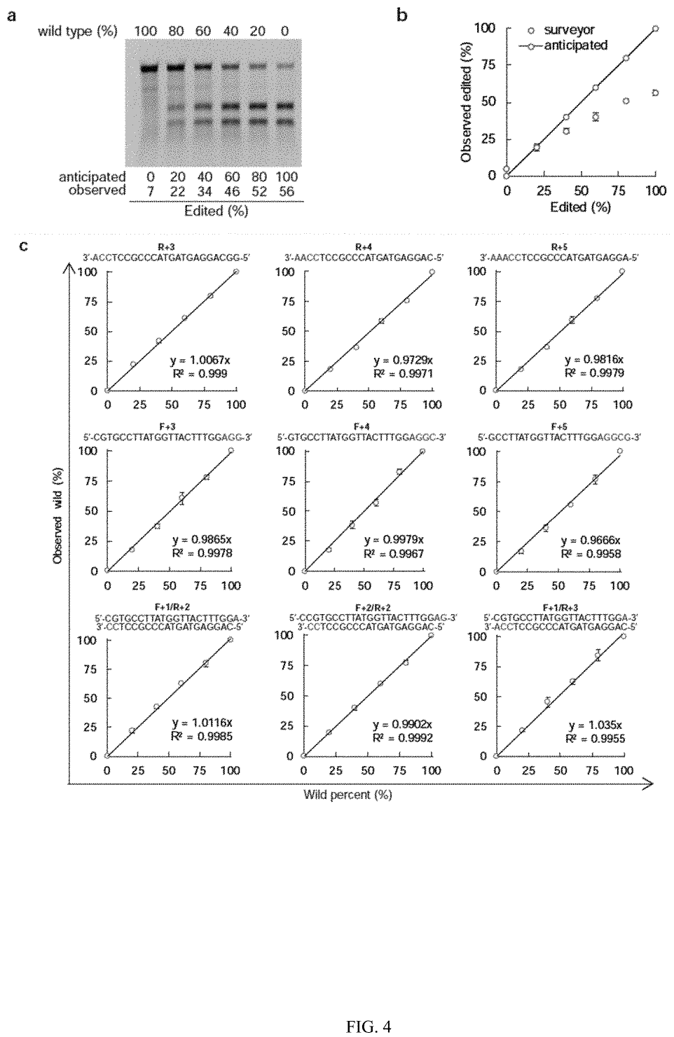

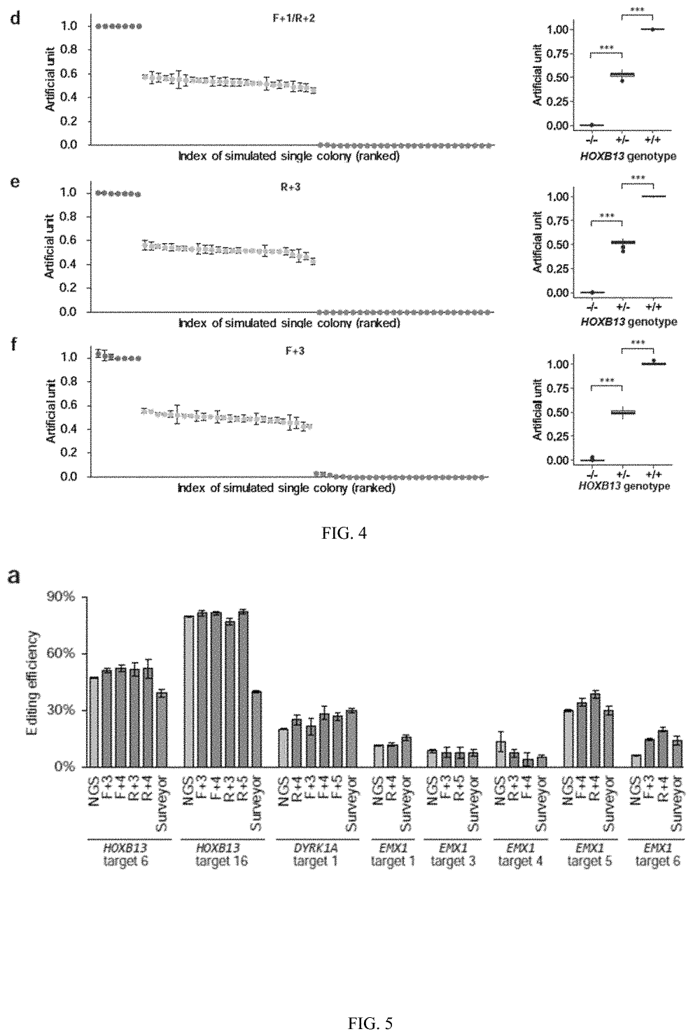

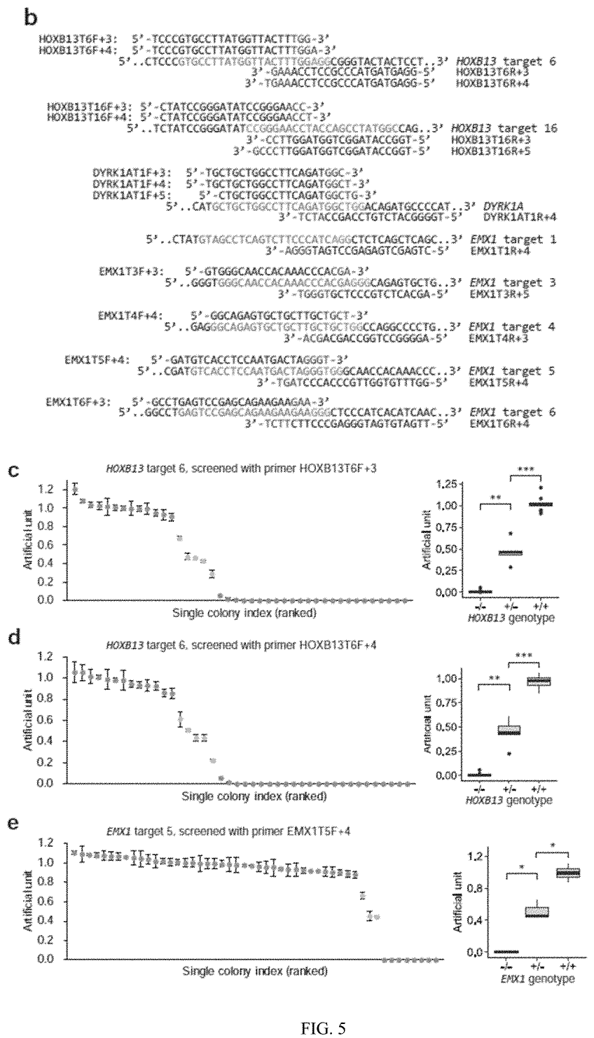

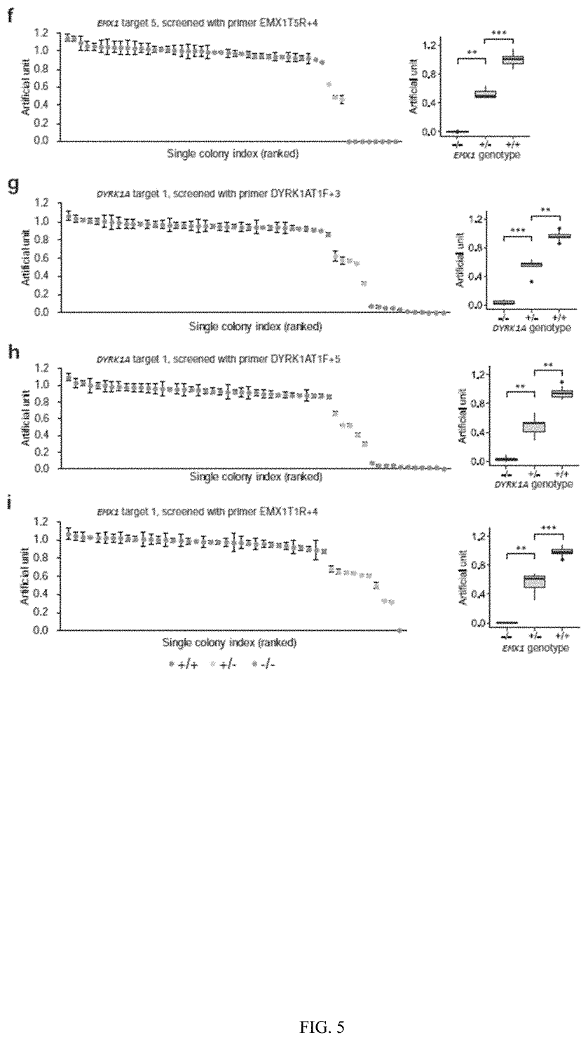

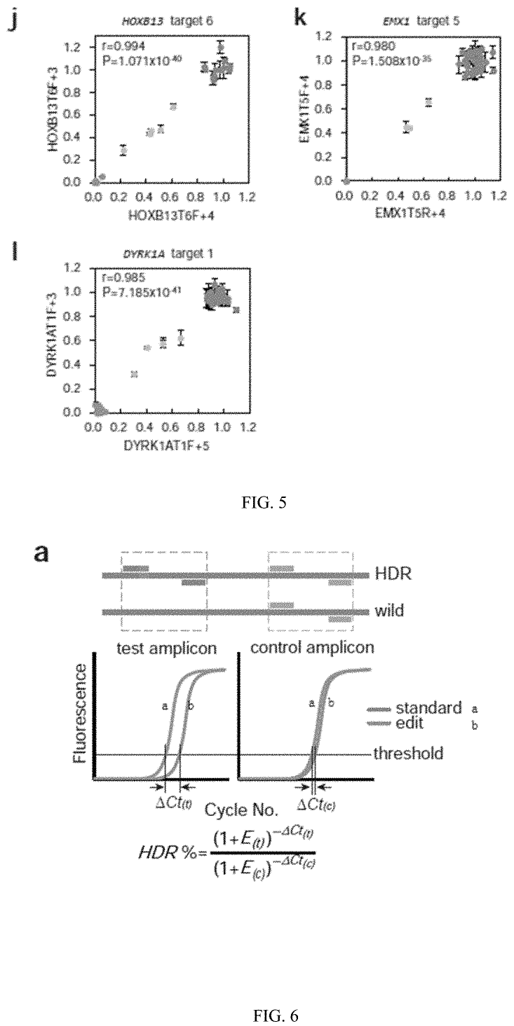

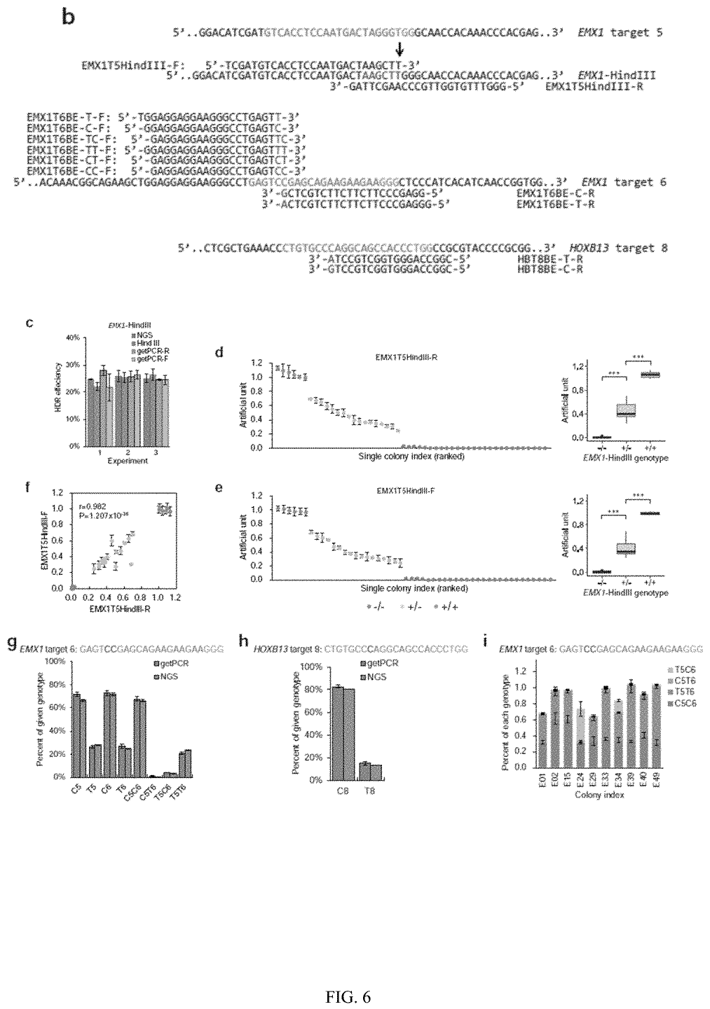

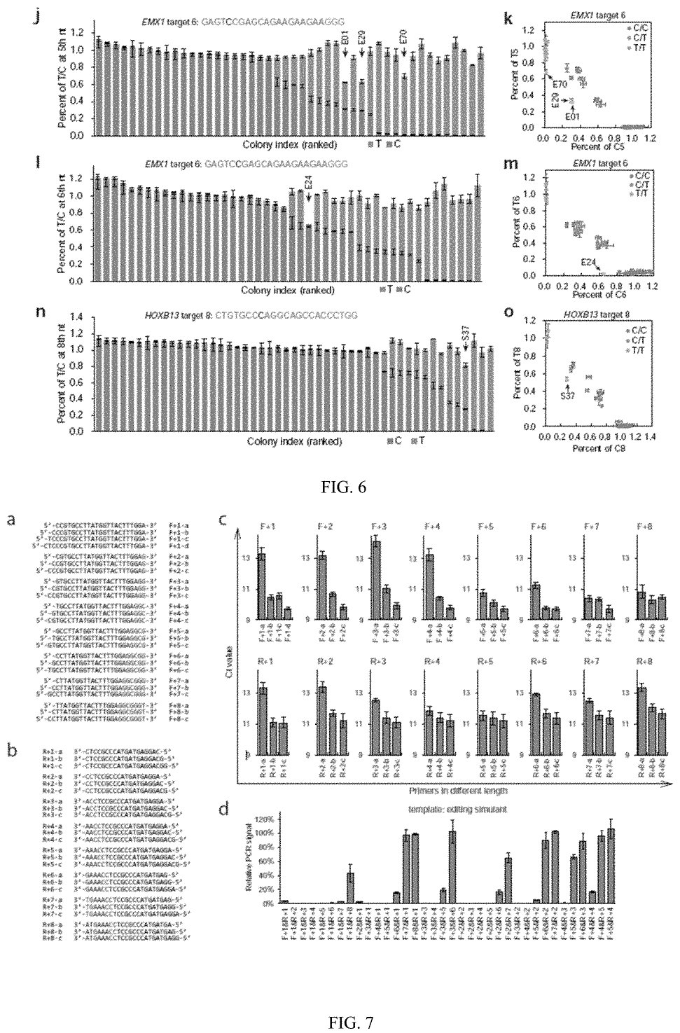

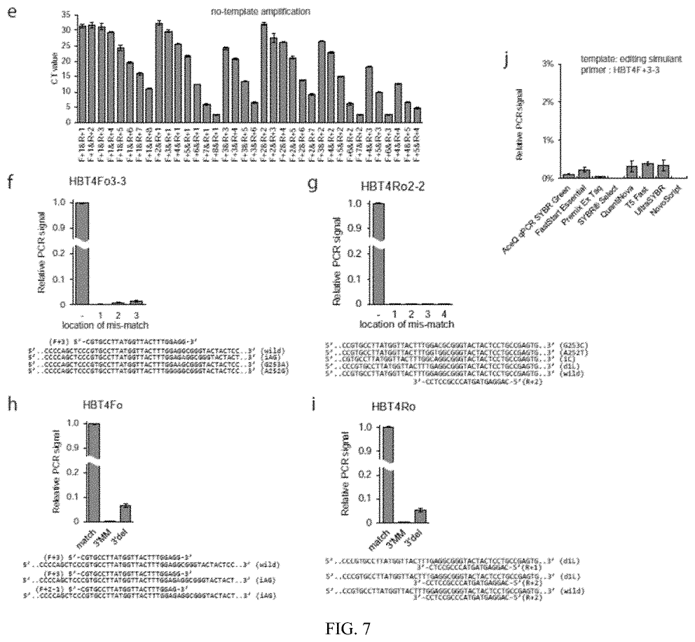

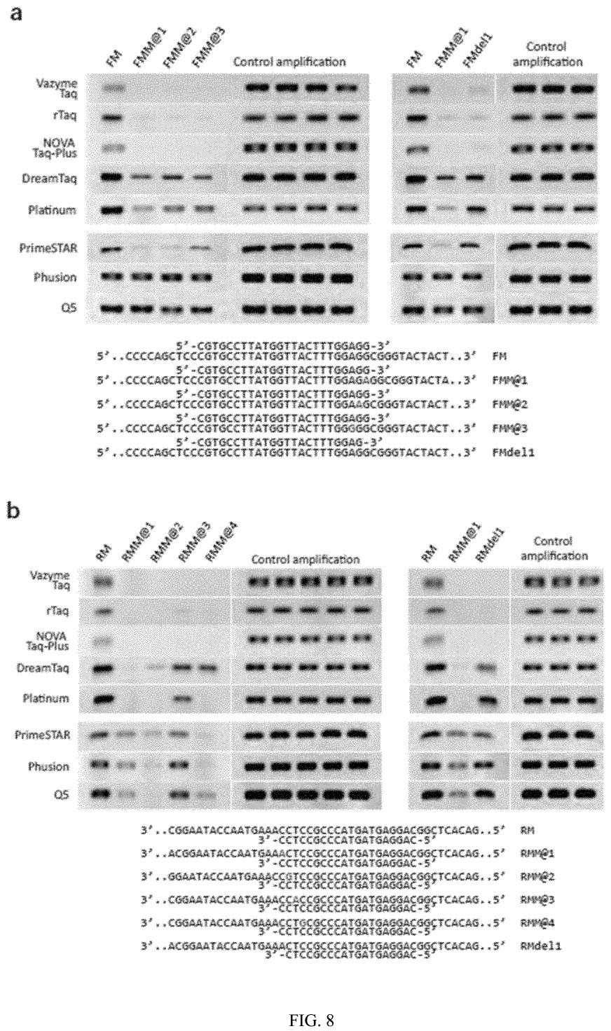

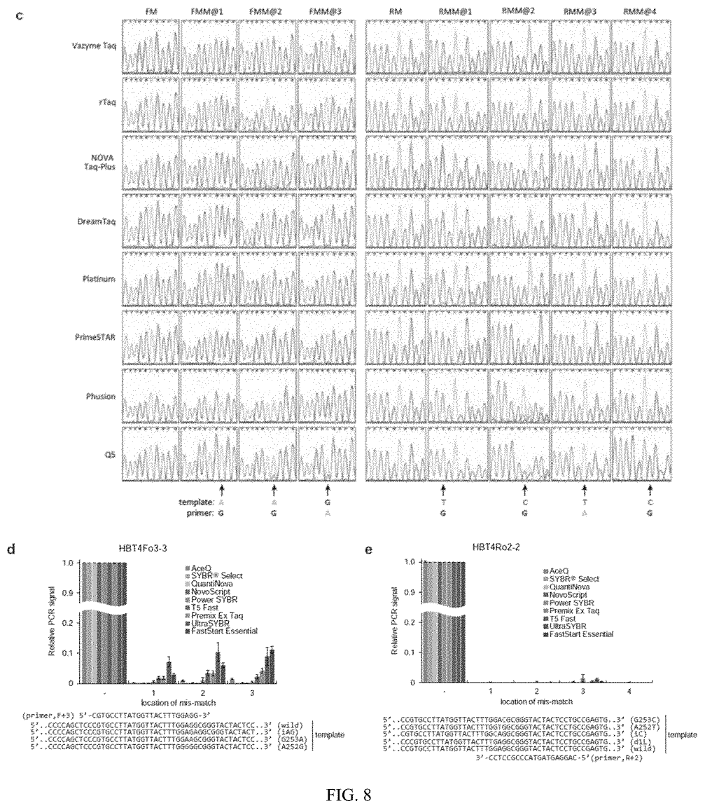

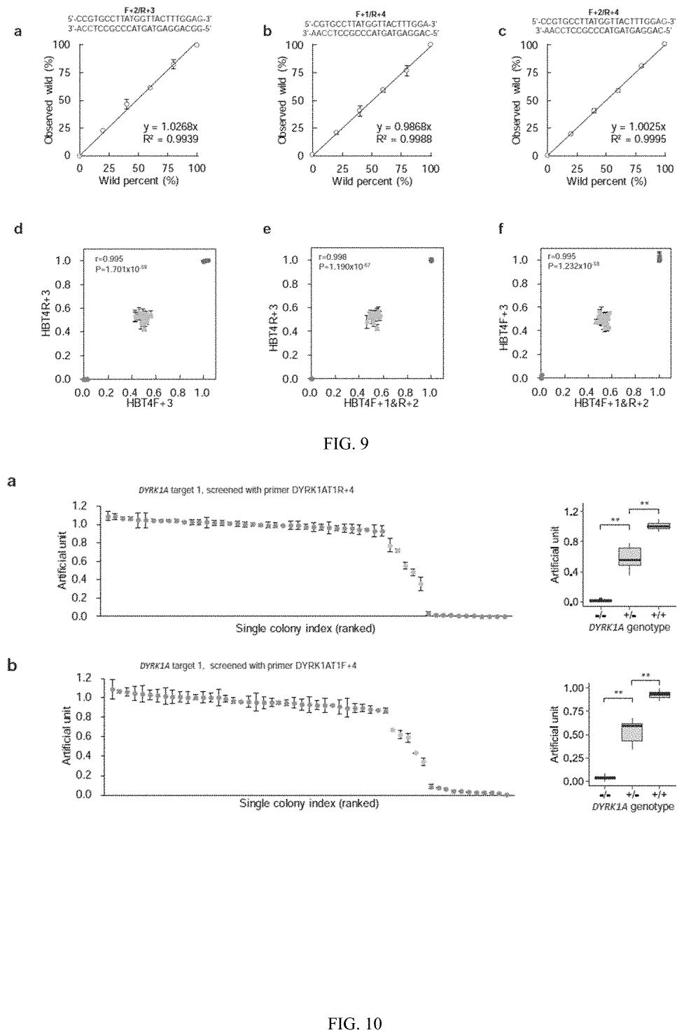

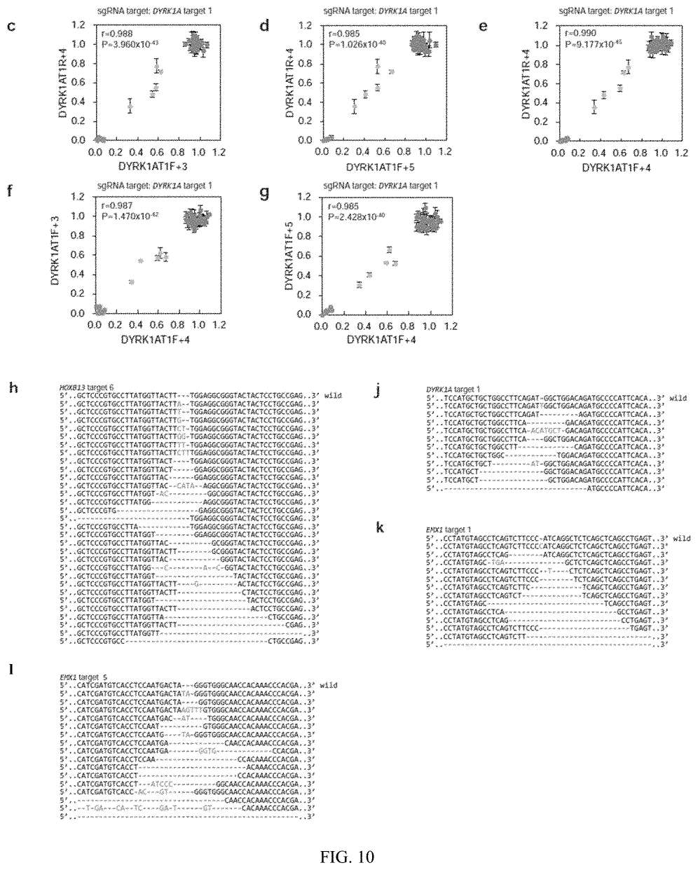

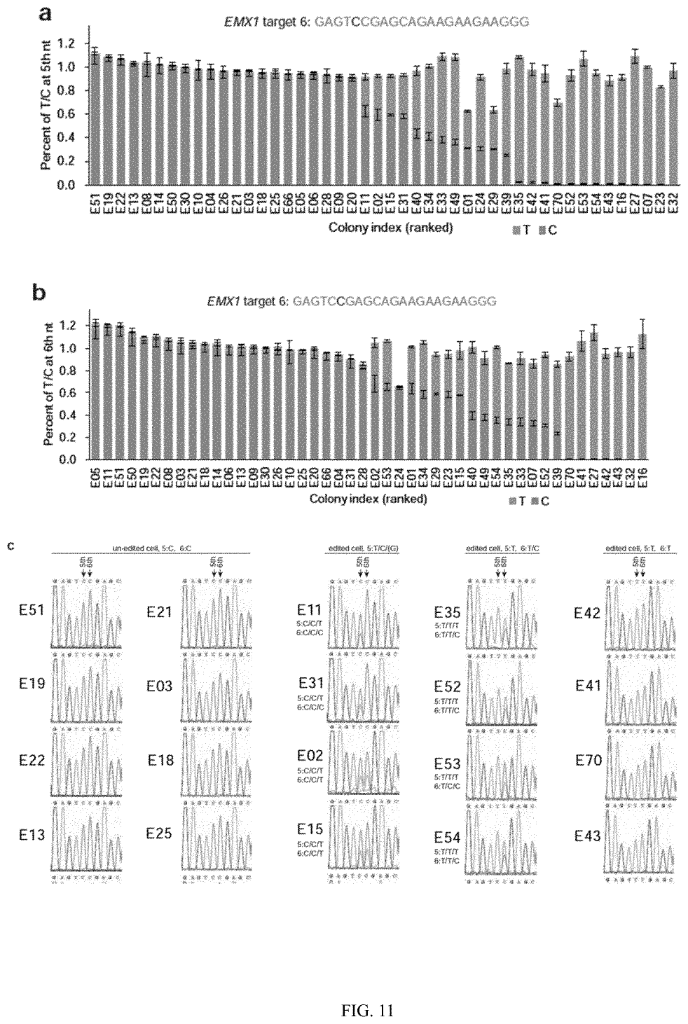

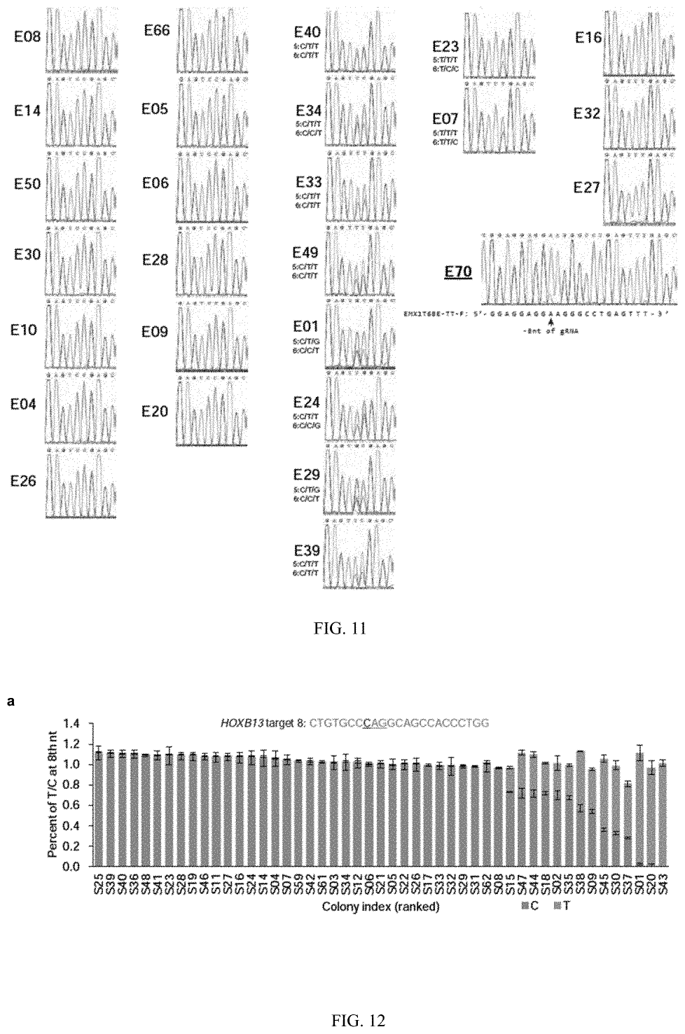

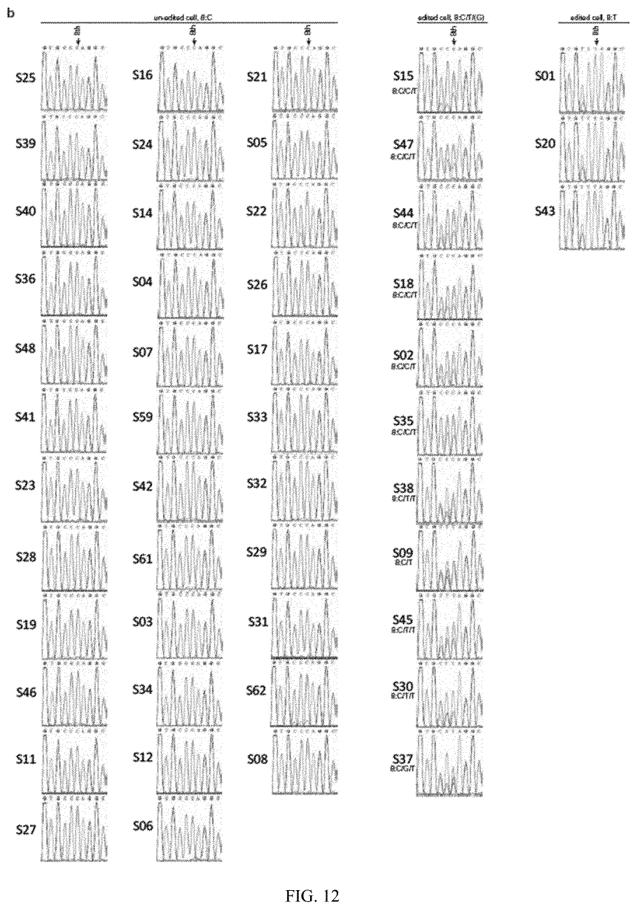

(d) Bar chart showing PCR specificity of watching primer combinations with indel mimic plasmids as template, alternative exhibition of FIG. 2e.

(e) Bar chart showing PCR self-amplification signal of watching primer combinations without adding template, alternative exhibition of FIG. 2f.

(f, g) Influence of single-base mismatch position relative to 3′ end on the PCR amplification, forward and reverse watching primer respectively.

(h, i) Comparison of 3′ end base mismatch with 3′ end base deletion for their ability in hampering PCR amplification, forward and reverse watching primer respectively.

(j) Comparison of multiple qPCR SYBR green mix products for their suitability in getPCR application. (Means ±s.e.m, n=3 independent technical replicates).

FIG. 8: Performance of different DNA polymerase products in mismatch discrimination. (a, b) Electrophoresis chromatography showing PCR amplification level with different DNA polymerase products from templates with or without mismatch base, forward and reverse watching primer respectively.

(c) Sanger sequencing chromatography of PCR products from a and b. (d, e) Bar chart illustrating sensitivity of multiple qPCR products to single-base mismatch at different position relative to 3′ end, with forward and reverse watching primer respectively. (Means±s.e.m, n=3 independent technical replicates).

FIG. 9: Editing frequency determination and single-cell colony genotyping with indel mimic plasmids.

(a-c) Frequency quantification of indel mimic DNA by getPCR method using forward and reverse watching primer in combination.

(d-f) Genotyping of mimic single-cell clones by combination of two differently designed getPCR watching primers. Referring to FIG. 2a for information of mimic indels. (Means±s.e.m, n =3 independent technical replicates).

FIG. 10: Single-cell colony genotyping for indels induced by gRNAs targeting HOXB13, DYRK1A and EMX1 genes.

(a, b) Genotyping of single-cell clones coming from edited 293T cells targeting DYRK1A gene through getPCR method with two differently designed watching primer respectively. Box plots show amplification quartile, median and third quartile, respectively, whiskers indicating 1.5 IQR, and outliers shown separately.

(c-g) Scatterplots showing the correlation and combination effect of two differently designed watching primers in genotyping.

(h-1) Illustration of indels discovered in single-cell clone genotyping by Sanger sequencing, for gRNA HOXB13 target 6, EMX1 target 5, DYRK1A targetl and EMX1 target1 respectively (Means±s.e.m, n=3 independent technical replicates, *P<0.05, **P<0.01, ***P<0.001).

FIG. 11: Single-cell colony genotyping in base editing by gRNAs targeting EMX1 gene. (a) Bar chart showing single-cell clone genotyping at 5^{th} nucleotide by getPCR in EMX1 gene base editing experiment, i.e., FIG. 6j annotated with detailed clone number.

(b) Bar chart showing single-cell clone genotyping at 6^{th} nucleotide by getPCR in EMX1 gene base editing experiment, i.e., FIG. 61 annotated with detailed clone number.

(c) Sanger sequencing chromatography in genotyping of single-cell clone. (Means±s.e.m, n =3 independent technical replicates).

FIG. 12: Single-cell colony genotyping for base-editing introduced stop codon on HOXB13 gene.

(a) Bar chart showing single-cell clone genotyping at 8^{th} nucleotide by getPCR in HOXB13 gene base editing experiment, i.e., FIG. 6n annotated with detailed clone number.

(b) Sanger sequencing chromatography in genotyping of single-cell clone. (Mean±s.e.m, n =3 independent technical replicates).

### DETAILED DESCRIPTION

It should be noted that the following detailed descriptions are exemplary and are intended to provide further illustration of the present application. Unless otherwise indicated, all technical and scientific terms used herein have the same meaning as commonly understood by one of ordinary skill in the art to which the present application belongs.

It is noted that the terms used herein are intended to describe specific embodiments only and are not intended to limit exemplary embodiments according to the present application. As used herein, unless the context clearly indicates otherwise, the singular form is also intended to include the plural form, and it is also to be understood that when the terms “comprising” and/or “including” are used in this specification, they indicate the presence of features, steps, operations, devices, components, and/or components , operations, devices, components, and/or combinations thereof.

As described in the background, prior art methods for detecting the efficiency of gene editing methods have certain drawbacks, such as Sanger, NGS, mismatch-specific nuclease based methods, which have the drawbacks of complicated operation, high cost, and lack of detection accuracy. It is important to provide a method that can be quickly, simply and reliably applied for quantification of genome editing efficiency and high-throughput genotyping without the need for a specific device. To achieve this technical purpose, the present application provides a getPCR assay method that uses the specificity of Taq polymerase to design primer sequences covering nuclease cut sites using wild-type DNA sequences as templates, and indirectly determines the editing efficiency of the genome by amplifying the percentage of wild-type DNA in the quantitative genome. After optimization and verification, the method has high detection accuracy and is easy to operate, and has a wide range of application values.

In order to enable those skilled in the art to understand the technical solutions of the present application more clearly, the technical solutions of the present application will be described in detail below in conjunction with specific embodiments and comparative examples.

The sources of reagents and materials used in the following embodiments are as follows.

Plasmids and oligos. The plasmid containing HOXB13 gene coding region in pcDNA3.1 vector was gifted by professor GH Wei from University of Oulu.

Twenty-six DNA variants simulating different potential indels at HOXB13 gRNA target 1 (FIG. 2a) and other 15 variants containing mutations to introduce different types of primer-template mismatches were constructed through site-directed mutagenesis. The sgRNA expression plasmid was constructed by deleting Cas9 expression cassette from pSpCas9 (BB) vector (Addgene, #42230) through PCR method. To construct plasmids expressing sgRNAs, annealed oligo pairs bearing 20-nt guide sequences were ligated into the sgRNA expression plasmid or the pSpCas9 (BB) vector between Bbsl sites. The high-fidelity CRISPR-Cas9 nuclease (R661A/Q695A/Q926A/D1135E) was obtained through site-directed mutagenesis on the basis of pSpCas9 (BB).

BE4-Gam plasmid (Addgene, #100806) was used for base editing experiments. The 99-nt single strand HDR template containing EMX1-HindIII mutation neighbor to the PAM sequence of EMX1 gRNA target 5 were synthetized in Invitrogen Trading (Shanghai) Co. Ltd. The EMx1 gene containing HindIII variation was also cloned into a plasmid and used as 100% HDR efficiency. Sequences of all the used primers and oligos are shown in Table 1.

TABLE 1
Oligos used in plasmid construction and transfection
a. Primers for construction of HOXB13 variant

| Plasmid | Primer 1 sequence (5me-35m | Primer 2 sequence (5im-3im | Template |
|---|---|---|---|
| HB251d1L | AGGCGGGTACTACTCCTGC | CAAAGTAACCATAAGGCACGG | OE1 |
| HB251d2L | AGGCGGGTACTACTCCTGC | AAAGTAACCATAAGGCACGGG | OE1 |
| HB251d3L | AGGCGGGTACTACTCCTGC | AAGTAACCATAAGGCACGGGA | OE1 |
| HB251d5L | AGGCGGGTACTACTCCTGC | GTAACCATAAGGCACGGGAGC | OE1 |
| HB251d10L | AGGCGGGTACTACTCCTGC | CATAAGGCACGGGAGCTGG | OE1 |
| HB251d15L | AGGCGGGTACTACTCCTGC | GGCACGGGAGCTGGGGACG | OE1 |
| HB251d1R | GGCGGGTACTACTCCTGCC | CCAAAGTAACCATAAGGCACG | OE1 |
| HB251d2R | GCGGGTACTACTCCTGCCG | CCAAAGTAACCATAAGGCACG | OE1 |
| HB251d3R | CGGGTACTACTCCTGCCGA | CCAAAGTAACCATAAGGCACG | OE1 |
| HB251d5R | GGTACTACTCCTGCCGAGTGT | CCAAAGTAACCATAAGGCACG | OE1 |
| HB251d10R | TACTCCTGCCGAGTGTCCC | CCAAAGTAACCATAAGGCACG | OE1 |
| HB251d15R | CTGCCGAGTGTCCCGGAGC | CCAAAGTAACCATAAGGCACG | OE1 |
| HB251d1L1R | GGCGGGTACTACTCCTGCC | CAAAGTAACCATAAGGCACGG | OE1 |
| HB251d2L2R | GCGGGTACTACTCCTGCCG | AAAGTAACCATAAGGCACGGG | OE1 |
| HB251d2L3R | CGGGTACTACTCCTGCCGA | AAAGTAACCATAAGGCACGGG | OE1 |
| HB251d2L5R | GGTACTACTCCTGCCGAGTGT | AAAGTAACCATAAGGCACGGG | OE1 |
| HB251d2L10R | TACTCCTGCCGAGTGTCCC | AAAGTAACCATAAGGCACGGG | OE1 |
| HB251iA | AAGGCGGGTACTACTCCTGC | CCAAAGTAACCATAAGGCACG | OE1 |
| HB251iT | TAGGCGGGTACTACTCCTGC | CCAAAGTAACCATAAGGCACG | OE1 |
| HB251iG | GAGGCGGGTACTACTCCTGC | CCAAAGTAACCATAAGGCACG | OE1 |
| HB25liC | CAGGCGGGTACTACTCCTGC | CCAAAGTAACCATAAGGCACG | OE1 |
| HB251iAA | AAAGGCGGGTACTACTCCTGC | CCAAAGTAACCATAAGGCACG | OE1 |
| HB251iAT | ATAGGCGGGTACTACTCCTGC | CCAAAGTAACCATAAGGCACG | OE1 |
| HB251iAG | AGAGGCGGGTACTACTCCTGC | CCAAAGTAACCATAAGGCACG | OE1 |
| HB251iAC | ACAGGCGGGTACTACTCCTGC | CCAAAGTAACCATAAGGCACG | OE1 |
| HB251iAAA | AAAAGGCGGGTACTACTCCTGC | CCAAAGTAACCATAAGGCACG | OE1 |
| 1MM-G | GGCGGGTACTACTCCTGCCG | CCCAAAGTAACCATAAGGCACG | OE1 |
| 2MM-G | GGCGGGTACTACTCCTGCCG | CTCAAAGTAACCATAAGGCACG | OE1 |
| 3MM-G | GGCGGGTACTACTCCTGCCG | CTTAAAGTAACCATAAGGCACG | OE1 |
| 1MM-C | GGCGGGTACTACTCCTGCCG | CCCAAAGTAACCATAAGGCACG | OE1 |
| 2MM-C | GGCGGGTACTACTCCTGCCG | CTCAAAGTAACCATAAGGCACG | OE1 |
| 3MM-C | CGCGGGTACTACTCCTGCCG | CTTAAAGTAACCATAAGGCACG | OE1 |
| 1MM-A | TGCGGGTACTACTCCTGCCG | CCCAAAGTAACCATAAGGCACG | OE1 |
| 2MM-A | TGCGGGTACTACTCCTGCCG | CTCAAAGTAACCATAAGGCACG | OE1 |
| 3MM-A | TGCGGGTACTACTCCTGCCG | CTTAAAGTAACCATAAGGCACG | OE1 |
| 1MM-T | AGCGGGTACTACTCCTGCCG | CCCAAAGTAACCATAAGGCACG | OE1 |
| 2MM-T | AGCGGGTACTACTCCTGCCG | CTCAAAGTAACCATAAGGCACG | OE1 |
| 3MM-T | AGCGGGTACTACTCCTGCCG | CTTAAAGTAACCATAAGGCACG | OE1 |
| A252T | cttatggttactttggTggcgggtactactcc | GGAGTAGTACCCGCCACCAAAG | OE1 |
| | | TAACCATAAG | |
| G253C | cttatggttactttggAcgcgggtactactcc | GGAGTAGTACCCGCGTCCAAAG | 1MM-C |
| | | TAACCATAAG | |
| G253A | cttatggttactttggAagcgggtactactcc | GGAGTAGTACCCGCTTCCAAAG | IMM-A |
| | | TAACCATAAG | |

b. Primers for construction of blank sgRNA expression plasmid

| Name | Primer 1 sequence (5′-3′) | Primer 2 sequence (5′-3′) | |
|---|---|---|---|
| sgRNA | TCCGGGAGCTGCATGTGTCA | GGGTACCTCTAGAGCCATTTG | addgene#42230 |
| expression | | | |
| plasmid | | | |

c. Primers for construction of HF-Cas9 (R661A, Q695A, Q926A, D1135E) through site-directed
mutagenesis

| Cas9 | | |
|---|---|---|
| variation | Primer 1 sequence (5′-3′) | Primer 2 sequence (5′-3′) |
| Cas9R661A | ACCGGCTGGGGCGCGCTGAGCCGGAA | CTTCCGGCTCAGCGCGCCCCAGCCG |
| | G | GT |
| Cas9Q695A | CAACAGAAACTTCATGGCGCTGATCCA | GTCGTCGTGGATCAGCGCCATGAAG |
| | CG | TTT |
| | ACGAC | CTGTTGG |
| Cas9Q926A | CTGGTGGAAACCCGGGCGATCACAAA | CACGTGCTTTGTGATCGCCCGGGTTT |
| | GC | CC |
| | ACGTG | ACCAG |
| Cas9D1135E | ACGGCGGCTTCGATAGCCCCACCGTGG | CCACGGTGGGGCTATCGAAGCCGCC |
| | | GT |

d. Primers for construction of sgRNA expression plasmids of given targets

| Target for | | |
|---|---|---|
| CRISPR | Primer 1 sequence (5′-3′) | Primer 2 sequence (5′-3′) |
| HOXB13 target 4 | CACCGCCTTATGGTTACTTTGGAGG | AAACCCTCCAAAGTAACCATAAGGC |
| HOXB13 target 6 | CACCGTGCCTTATGGTTACTTTGG | AAACCCAAAGTAACCATAAGGCAC |
| HOXB13 target 16 | CACCGCCATAGGCTGGTAGGTTCC | AAACGGAACCTACCAGCCTATGGC |
| HOXB13 target 8 | CACCGCTGTGCCCAGGCAGCCACCC | AAACGGGTGGCTGCCTGGGCACAG |
| | | C |
| DYRK1A target 1 | CACCGGCTGCTGGCCTTCAGATGGC | AAACGCCATCTGAAGGCCAGCAGC |
| | | C |
| EMX1 target 1 | CACCGGTAGCCTCAGTCTTCCCATC | AAACGATGGGAAGACTGAGGCTAC |
| | | C |
| EMX1 target 3 | CACCGGGGCAACCACAAACCCACG | AAACTCGTGGGTTTGTGGTTGCCCC |
| | A | |
| EMX1 target 4 | CACCGGGCAGAGTGCTGCTTGCTGC | AAACGCAGCAAGCAGCACTCTGCC |
| | | C |
| EMX1 target 5 | CACCGGTCACCTCCAATGACTAGGG | AAACCCCTAGTCATTGGAGGTGACC |
| EMX1 target 6 | CACCGGAGTCCGAGCAGAAGAAGA | AAACTTCTTCTTCTGCTCGGACTCC |
| | A | |

e. HDR template sequence (5′-3′)

| | |
|---|---|
| EMX1-HindIII | cacgaagcaggccaatggggaggacatcgatgtcacctccaatgactAAGCTT |
| | gggcaaccacaaacccacgagggcagtgctgcttgctgctggcc |

Cell culture The Lenti-X 293 T cells (Cat #632180) was originally purchased from Clontech Laboratories Inc. and cultured in Dulbecco's Modified Eagle's Medium (Gibco, Cat #C11995500BT) supplemented with 1×penicillin/streptomycin (HyClone, Cat #SV30010) and 10% (v/v) FBS (Gibco, Cat#10270-106), at 37° C. with 5% CO₂. It was checked regularly for mycoplasma using MycoBlueTM Mycoplasma Detector kit according to product manual (Vazyme, Cat #D101-01). The cell line was proven to be mycoplasma free during our study.

Transfections The Lenti-X 293 T cells were seeded into 24-well plates (Labserv, Cat #310109007) at a density of 120,000 cells per well the day before transfection. Cells were transfected at ˜70% confluency using Lipofectamine 2000 (TermoFisher Scientifc, Cat #11668019) according to the manufacturer's instruction. For indel detection, 1 μg of plasmid that expressing both sgRNA and high-fidelity CRISPR-Cas9 was applied in each transfection. For base editing, 750 ng of BE4 plasmid and 250 ng of sgRNA expression plasmid were used for each transfection reaction. For HDR-mediated genome modification, 600 ng of plasmid that expressing both sgRNA and high-fidelity CRISPR-Cas9 as well as 10 pmol HDR oligo were used for each transfection. 48 h After transfection, genomic DNA was extracted with a TIANamp Genomic DNA Kit (TIANGEN, Cat#DP304-03) according to the manufacturer's instruction.

getPCR conditions. For each getPCR reaction, 0.1 ng of plasmid DNA or 2.5 ng of genomic DNA was used as template in 15 μL reaction system of AceQ qPCR SYBR Green Master Mix (Vazyme, Cat #Q111-02). Real-time PCR was run on the thermocyclers Rotor-Gene Q (Qiagen, Germany) using the following program: initial denaturation at 95° C. for 5 min, then 40 cycles at 95° C. for 30 s, 65-69° C. for 30 s and at 72° C. for 10 s with fluorescence acquirement. While employing LightCycler® 96 Thermal cycler Instrument (Roche Applied Science, Germany), the following conditions were used: 40 cycles at 95° C. for 15 s, 65-69° C. for 20 s and 72° C. for 10 s with fluorescence acquirement, followed by a standard melting curve stage. The primer Tm value is calculated using the online Oligo Calc tool.

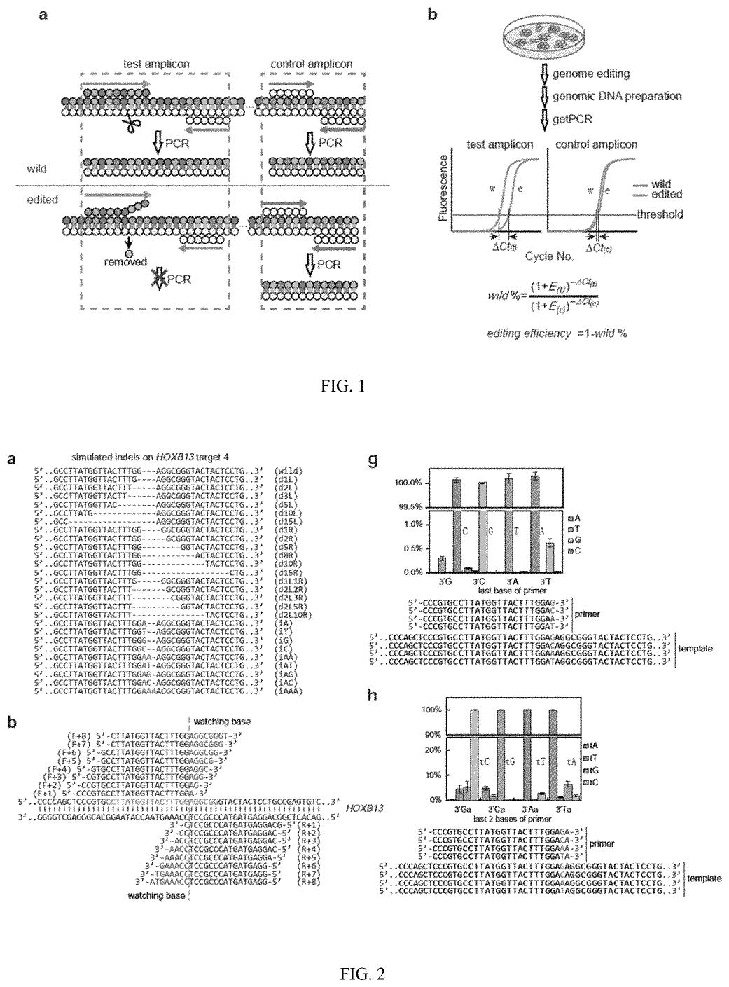

Indel frequency quantification using getPCR. The 26 plasmids mimicking different type of indels were mixed equally and regarded as 100% indels (FIG. 2a), which can be mixed further with wild type DNA at given ratio to obtain DNA samples with diverse indel frequencies. The indel frequencies were evaluated using getPCR method. In getPCR assay, 0.1 ng of plasmid DNA was used as template for each qPCR reaction. The wild type percentage in the mixture sample and indel frequency were calculated as described in FIG. 1b. Simultaneously, each of these 26 plasmids was used to simulate single-cell clones with homozygous indel. Each plasmid was also equally mixed with wild type DNA plasmid to simulate heterozygous single-cell clones that bearing indel on one allele. Sequences of the getPCR primers are shown in Table 2. As to indel frequency quantification on genomic DNA sample, 2.5 ng of genomic DNA was included as template and amplified using primer as summarized in Table 3.

TABLE 2
Genome editing efficiency determination
a. Primers for Surveyor DNA amplification and sanger sequencing

| Gene | Primer 1 sequence (5′-3′) | Primer 2 sequence (5′-3′) |
|---|---|---|
| HOXB13 | CCGGCAATTATGCCACCTTG | GGTGGGTTCTGTTCTCCCTG |
| DYRK1A | GGAGCTGGTCTGTTGGAGAA | TCCCAATCCATAATCCCACGTT |
| EMX1 | CCATCCCCTTCTGTGAATGT | GGAGATTGGAGACACGGAGA |

b. Primers for getPCR in detecting indels atHOXB13 target 4 site

| Primer ID | Sequence (5′-3′) | |
|---|---|---|
| universal-F | CCTGGGGTGCCCCAGGGGAC | Forward primer |
| R + 1 | GCAGGAGTAGTACCCGCCTC | watching primer-R |
| R + 2 | CAGGAGTAGTACCCGCCTCC | watching primer-R |
| R + 3 | CAGGAGTAGTACCCGCCTCCA | watching primer-R |
| R + 4 | CAGGAGTAGTACCCGCCTCCAA | watching primer-R |
| R + 5 | AGGAGTAGTACCCGCCTCCAAA | watching primer-R |
| R + 6 | GGAGTAGTACCCGCCTCCAAAG | watching primer-R |
| R + 7 | GGAGTAGTACCCGCCTCCAAAGT | watching primer-R |
| R + 8 | GGAGTAGTACCCGCCTCCAAAGTA | watching primer-R |
| universal-R | GGGGCGGCTGGGGTACTCTTC | Forward primer |
| F + 1 | CCCGTGCCTTATGGTTACTTTGGA | watching primer-F |
| F + 2 | CCGTGCCTTATGGTTACTTTGGAG | watching primer-F |
| F + 3 | CGTGCCTTATGGTTACTTTGGAGG | watching primer-F |
| F + 4 | GTGCCTTATGGTTACTTTGGAGGC | watching primer-F |
| F + 5 | GCCTTATGGTTACTTTGGAGGCG | watching primer-F |
| F + 6 | GCCTTATGGTTACTTTGGAGGCGG | watching primer-F |
| F + 7 | CTTATGGTTACTTTGGAGGCGGG | watching primer-F |
| F + 8 | CTTATGGTTACTTTGGAGGCGGGT | watching primer-F |
| universal-R | GGGGCGGCTGGGGTACTCTTC | Forward primer |
| F + 3-1 | TGCCTTATGGTTACTTTGGAGG | watching primer-F |
| F + 3-2 | GTGCCTTATGGTTACTTTGGAGG | watching primer-F |
| F + 3-3 | CGTGCCTTATGGTTACTTTGGAGG | watching primer-F |
| F + 3-4 | CCGTGCCTTATGGTTACTTTGGAGG | watching primer-F |
| F + 3-5 | CCCGTGCCTTATGGTTACTTTGGAGG | watching primer-F |
| F + 4-1 | GCCTTATGGTTACTTTGGAGGC | watching primer-F |
| F + 4-2 | TGCCTTATGGTTACTTTGGAGGC | watching primer-F |
| F + 4-3 | GTGCCTTATGGTTACTTTGGAGGC | watching primer-F |
| F + 4-4 | CGTGCCTTATGGTTACTTTGGAGGC | watching primer-F |
| F + 4-5 | CCGTGCCTTATGGTTACTTTGGAGGC | watching primer-F |
| universal-F | CCTGGGGTGCCCCAGGGGAC | Forward primer |
| R + 4-1 | GGAGTAGTACCCGCCTCCAA | watching primer-R |
| R + 4-2 | AGGAGTAGTACCCGCCTCCAA | watching primer-R |
| R + 4-3 | CAGGAGTAGTACCCGCCTCCAA | watching primer-R |
| R + 4-4 | GCAGGAGTAGTACCCGCCTCCAA | watching primer-R |
| R + 4-5 | GGCAGGAGTAGTACCCGCCTCCAA | watching primer-R |
| R + 3-1 | GGAGTAGTACCCGCCTCCA | watching primer-R |
| R + 3-2 | AGGAGTAGTACCCGCCTCCA | watching primer-R |
| R + 3-3 | CAGGAGTAGTACCCGCCTCCA | watching primer-R |
| R + 3-4 | GCAGGAGTAGTACCCGCCTCCA | watching primer-R |
| R + 3-5 | GGCAGGAGTAGTACCCGCCTCCA | watching primer-R |
| universal-R | CAGTGGGGCGGCTGGGGTA | Reverse primer |
| 253-G | CCCGTGCCTTATGGTTACTTTGGAG | watching primer-F |
| 253-C | CCCGTGCCTTATGGTTACTTTGGAC | watching primer-F |
| 253-A | CCCGTGCCTTATGGTTACTTTGGAA | watching primer-F |
| 253-T | CCCGTGCCTTATGGTTACTTTGGAT | watching primer-F |

TABLE 3
In-cell genome editing efficiency
a. getPCR primers for indel efficiency quantification

| | getPCR | Primer 1 sequence (5′-3′) | |
|---|---|---|---|
| Target site | Primers | (Watching primer) | Primer 2 sequence (5′-3′) |
| HOXB13 | HBT16Fo3 | ctatccgggatatccgggaacc | GAGTCTGCACCACAGACACG |
| target 16 | | | TCC |
| HOXB13 | HBT16Fo4 | ctatccgggatatccgggaacct | GAGTCTGCACCACAGACACG |
| target 16 | | | TCC |
| HOXB13 | HBT16Ro3 | tggccataggctggtaggttcc | accccgcggagactcccacg |
| target 16 | | | |
| HOXB13 | HBT16Ro5 | ccataggctggtaggttcccg | accccgcggagactcccacg |
| target 16 | | | |
| HOXB13 | HBT6Fo3 | tcccgtgccttatggttactttgg | CAGTGGGGCGGCTGGGGTA |
| target 6 | | | |
| HOXB13 | HBT6Fo4 | tcccgtgccttatggttactttgga | CAGTGGGGCGGCTGGGGTA |
| target 6 | | | |
| HOXB13 | HBT6Ro3 | ggagtagtacccgcctccaaag | tggggtgccccaggggac |
| target 6 | | | |
| HOXB13 | HBT6Ro4 | ggagtagtacccgcctccaaagt | tggggtgccccaggggac |
| target 6 | | | |
| | Control | Primer 1 sequence (5′-3′) | |
| | Primers | | Primer 2 sequence (5′-3′) |
| | HB13-104ctrl | GCGACATGACTCCCTGTTGCCTGT | GACCTGGTGGGTTCTGTTCTC |
| | | G | CCTG |
| | getPCR | Primer 1 sequence (5′-3′) | |
| Target site | Primers | (Watching primer) | Primer 2 sequence (5′-3′) |
| DYRK1A | DY-Ro4 | tggggcatctgtccagccatct | ttgtaggaggagagacttcagcatgc |
| target 1 | | | |
| DYRK1A | DY-Fo3 | tgctgctggccttcagatggc | tatgataaggcagaaacctgttggtcac |
| target 1 | | | |
| DYRK1A | DY-Fo4 | tgctgctggccttcagatggct | tatgataaggcagaaacctgttggtcac |
| target 1 | | | |
| DYRK1A | DY-Fo5 | ctgctggccttcagatggctg | tatgataaggcagaaacctgttggtcac |
| target 1 | | | |
| | Control | | |
| | Primers | Primer 1 sequence (5′-3′) | Primer 2 sequence (5′-3′) |
| | DY-99ctrl | GCGATGTTGTTTGCCGTAAACCTG | GACTTCTCCAACAGACCAGCT |
| | | GC | CCTC |
| | getPCR | Primer 1 sequence (5′-3′) | |
| Target site | Primers | (Watching primer) | Primer 2 sequence (5′-3′) |
| EMX1 target | EMX1T1-R04 | CCATCCCCTTCTGTGAATGTtagaccc | ctgagctgagaGcctgatggga |
| 1 | | | |
| | Control | | |
| | Primers | Primer 1 sequence (5′-3′) | Primer 2 sequence (5′-3′) |
| | EMX1-113ctrl | CGATGTCACCTCCAATGACTAGGGT | CAGGGAGTGGCCAGAGTCCA |
| | | G | GCT |
| | getPCR | Primer 1 sequence (5′-3′) | |
| Target site | Primers | (Watching primer) | Primer 2 sequence (5′-3′) |
| EMX1 target | EMXI13-Fo3 | gtgggcaaccacaaacccacga | aggggcctggccagcagca |
| 3 | | | |
| EMX1 target | EMX1T3-R05 | agcactctgccctcgtgggt | gatgtcacctccaatgactagggt |
| 3 | | | |
| EMX1 target | EMX1T4-R03 | aggggcctggccagcagca | gtgggcaaccacaaacccacga |
| 4 | | | |
| EMX1 target | EMX1T4-Fo4 | ggcagagtgctgcttgctgct | TCCCCAAAGCCTGGCCAGGG |
| 4 | | | AGT |
| EMX1 target | EMX1T5-Fo4 | gatgtcacctccaatgactagggt | agcactctgccctcgtgggt |
| 5 | | | |
| EMX1 target | EMX1T5-R04 | ggtttgtggttgcccaccctagt | gcctgagtccgagcagaagaagaa |
| 5 | | | |
| EMX1 target | EMX1T6-Fo3 | gcctgagtccgagcagaagaagaa | ggtttgtggttgcccaccctagt |
| 6 | | | |
| EMX1 target | EMX1T6-Ro4 | ttgatgtgatgggagcccttcttct | gaggccccagtggctgctct |
| 6 | | | |
| | Control | | |
| | Primers | Primer 1 sequence (5′-3′) | Primer 2 sequence (5′-3′) |
| | EMXI-1llctrl | CCATCCCCTTCTGTGAATGTtagaccc | TGAGCTGAGAGCCTGATGGG |
| | | | AAGAC |

b. getPCR primers for base editing efficiency quantification

| | getPCR | Primer 1 sequence (5′-3′) | |
|---|---|---|---|
| Target site | Primers | (Watching primer) | Primer 2 sequence (5′-3′) |
| HOXB13 | C8 | cggccagggtggctgcctG | ccgtgccttatggttactttggagg |
| target 8 | | | |
| HOXB13 | T8 | cggccagggtggctgcctA | ccgtgccttatggttactttggagg |
| target 8 | | | |
| | Control | | |
| | Primers | Primer 1 sequence (5′-3′) | Primer 2 sequence (5′-3′) |
| | HB13-104ctrl | GCGACATGACTCCCTGTTGCCTGT | GACCTGGTGGGTTCTGTTCTC |
| | | G | CCTG |
| | getPCR | Primer 1 sequence (5′-3′) | |
| Target site | Primers | (Watching primer) | Primer 2 sequence (5′-3′) |
| EMX1 target | 5C | ggaggaggaagggcctgagtC | ggtttgtggttgcccaccctagt |
| 6 | | | |
| EMX1 target | 5T | tggaggaggaagggcctgagtT | ggtttgtggttgcccaccctagt |
| 6 | | | |
| EMX1 target | 6C | ggagcccttcttcttctgctcG | gaggccccagtggctgctct |
| 6 | | | |
| EMX1 target | 6T | gggagcccccttcttcttctgctcA | gaggccccagtggctgctct |
| 6 | | | |
| EMX1 target | CC | gaggaggaagggcctgagtCC | ggtttgtggttgcccaccctagt |
| 6 | | | |
| EMX1 target | TT | ggaggaggaagggcctgagtTT | ggtttgtggttgcccaccctagt |
| 6 | | | |
| EMX1 target | CT | gaggaggaagggcctgagtCT | ggtttgtggttgcccaccctagt |
| 6 | | | |
| EMX1 target | TC | gaggaggaagggcctgagtT C | ggtttgtggttgcccaccctagt |
| 6 | | | |
| | Control | | |
| | Primers | Primer 1 sequence (5′-3′) | Primer 2 sequence (5′-3′) |
| | EMXI-11lctrl | CCATCCCCTTCTGTGAATGTtagaccc | TGAGCTGAGAGCCTGATGGG |
| | | | AAGAC |

c. getPCR primers for HDR repairing efficiency quantification

| | getPCR | Primer 1 sequence (5′-3′) | |
|---|---|---|---|
| Target site | Primers | (Watching primer) | Primer 2 sequence (5′-3′) |
| EMX1 target | EMX1T5HindIII- | tcgatgtcacctccaatgactAAGCTT | agcactctgccctcgtgggt |
| 5 | F | | |
| EMX1 target | EMX1T305-R | gggtttgtggttgcccaAGCTTag | gcctgagtccgagcagaagaagaa |
| 5 | | | |
| | Control | | |
| | Primers | Primer 1 sequence (5′-3′) | Primer 2 sequence (5′-3′) |
| | EMX1-11lctrl | CCATCCCCTTCTGTGAATGTtagaccc | TGAGCTGAGAGCCTGATGGG |
| | | | AAGAC |

Surveyor nuclease assay. Indel frequencies were also determined using surveyor nuclease assay method with Surveyor® Mutation Detection Kits (Integrated DNA Technologies, Cat #706020) as described previously. In brief, genomic DNA was extracted using TIANamp Genomic DNA Kit (TIANGEN, Cat #DP304-03) according to product manual. DNA regions were then amplified with the cut site 200-400 bp away from each end using high-fidelity PrimeSTAR® Max DNA Polymerase (TaKaRa, Cat #R045B) and primers summarized in Table 2a. 270 ng of purified PCR product was subjected to heteroduplex formation using a T100™ Thermal Cycler (Bio-Rad) and subsequently treated with Surveyor Nuclease according to user guide. The DNA fragments were separated on 2% agarose gel and images were acquired using Quantum-ST5 (VILBER LOURMAT, France) and analyzed with Quantum ST5 Xpress software.

Application of getPCR in HDR and BE4 experiments. Variation-specific getPCR primers were designed with Modified nucleotide(s) at 3′ end as summarized in Table 3. In getPCR analysis, 2.5 ng of genomic DNA was included as template for each reaction. The genome modification efficiencies were calculated using the equation as shown in FIG. 6a.

HindIII-based RFLP assay. In the HDR experiments targeting EMX1 gene, one HindIII site was introduced neighbor to the PAM sequence, which enabled HDR efficiency quantification through HindIII-based restriction fragment length polymorphism (RFLP) analysis. Briefly, 639 bp of DNA region with HindIII site 355 bp away from 5′ end was amplified using PrimeSTAR® Max DNA Polymerase and primers same to Surveyor assay as shown in Table 2a and purified using Universal DNA Purification Kit (TIANGEN, Cat #DP214). 270 ng of PCR product was subjected to HindIII digestion and resolved on a 2% agarose gel. The images were acquired using Quantum-ST5 (VILBER LOURMAT, France) and analyzed with Quantum ST5 Xpress software.

NGS-based methods. DNA regions covering genome modification were amplified to construct NGS libraries and editing efficiencies were then calculated by counting the NGS reads. Sequencing libraries were prepared with two rounds of PCR amplifications with genomic DNA as template. In the first round PCR, amplicons of 250-280 bp were designed with the Cas9 cutting site near the middle part and the binding sites of Illumina sequencing primers were introduced at both ends. In the second round PCR, adaptors for cluster generation and index sequences were attached. After Purification and quantification, the libraries were subjected to 150 bp paired-end sequencing on the Illumina HiSeq X-TEN platform run by Genewiz. For NHEJ mediated indels, the wild type read counts in each library were acquired with wild type DNA sequence and the indel editing efficiency was calculated using the equation “Editing efficiency=1-wide_type_counts/total_counts*100%”. As to modification efficiency in base editing and HDR experiments, the read counts of expected DNA variation sequences in the library were acquired and editing efficiencies were calculated using the equation “Efficiency=expected_sequence_counts/total_counts*100%”. Full details of the library preparation and counting method can be found in Table 4.

TABLE 4
Genome editing efficiency quantification by NGS
a. Primers for library preparation

| | | Amplification Round Primer 1 | Amplification Round Primer |
|---|---|---|---|
| Target site | Primer ID | sequence (5′-3′) | sequence (5′-3′) |
| EMX1-T1 | EMX1-1lib | ACTCTTTCCCTACACGACGCT | GTGACTGGAGTTCAGAC |
| | | CTTCCGATCTccatccccttctgtgaatgttagacc | GTGTGCTCTTCCGATCT |
| | | | ccttcctcctccagcttctgccgt |
| EMX1-T2 | EMX1-1lib | ACTCTTTCCCACACGACGCT | GTGACTGGAGTTCAGA |
| | | CTTCCGATCTccatccccttctgtgaatgttagacc | CGTGTGCTCTTCCGATCT |
| | | | ccttcctcctccagcttctgccgt |
| EMX1-T3 | EMX1-2lib | ACTCTTTCCCTACACGACGCT | GTGACTGGAGTTCAGAC |
| | | CTTCCGATCTggttccagaaccggaggacaaagta | GTGTGCTCTTCCGATCT |
| | | c | gactccaggcctccccaaagcctg |
| EMX1-T4 | EMX1-2lib | ACTCTTTCCCTACACGACGCTC | GTGACTGGAGTTCAGAC |
| | | TTCCGATCTggttccagaaccggaggacaaagtac | GTGTGCTCTTCCGATCT |
| | | | gactccaggcctccccaaagcctg |
| EMX1-T5 | EMX1-2lib | ACTCTTTCCCTACACGACGCTC | GTGACTGGAGTTCAGAC |
| | | TTCCGATCTggttccagaaccggaggacaaagtac | GTGTGCTCTTCCGATCT |
| | | | gactccaggcctccccaaagcctg |
| EMX1-T6 | EMX1-2lib | ACTCTTTCCCTACACGACGCTC | GTGACTGGAGTTCAGAC |
| | | TTCCGATCTggttccagaaccggaggacaaagtac | GTGTGCTCTTCCGATCT |
| | | | gactccaggcctccccaaagcctg |
| EMX1-T6- | EMX1-2lib | ACTCTTTCCCTACACGACGCTC | GTGACTGGAGTTCAGAC |
| BE4 | | TTCCGATCTggttccagaaccggaggacaaagtac | GTGTGCTCTTCCGATCT |
| | | | gactccaggcctccccaaagcctg |
| EMX1-5 with | EMX1-3lib | ACTCTTTCCCTACACGACGCTC | GTGACTGGAGTTCAGAC |
| HindIII | | TTCCGATCTgtggttccagaaccggaggacaaagta | GTGTGCTCTTCCGATCT |
| | | | ctccaggcctccccaaagcctggc |
| DYRK1A | DYRK1Alib | ACTCTTTCCCTACACGACGCTC | GTGACTGGAGTTCAGA |
| | | TTCCGATCTggatatgaatatttcctttaaacctcac | CGTGTGCTCTTCCGATC |
| | | | ttccatgaacttacctggttagttag |
| HOXB13-T6 | HB13-251lib | ACTCTTTCCCTACACGACGCTC | GTGACTGGAGTTCAGA |
| | | TTCCGATCTctgtcaactatgcccccttggatctg | CGTGTGCTCTTCCGATC |
| | | | tggcaaactcagtggggcggctgg |
| HOXB13-T8 | HB13-251lib | ACTCTTTCCCTACACGACGCTC | GTGACTGGAGTTCAGA |
| (BE4) | | TTCCGATCTctgtcaactatgcccccttggatctg | CGTGTGCTCTTCCGATC |
| | | | tgttccagccaccagagcgagccca |
| HOXB13- | HB13-404lib | ACTCTTTCCCTACACGACGCTC | GTGACTGGAGTTCAGA |
| T16 | | TTCCGATCTcccggagctcgctgaaaccctgtg | CGTGTGCTCTTCCGATC |
| | | | tgttccagccaccagagcgagccca |

1st round PCR, take 50 ng gDNA as template, 28 cycles, 15 μl system, set NTC control, anealed
@ 60° C., using PrimeSTAR® Max DNA Polymerase (TaKaRa)

| Second | | |
|---|---|---|
| Round | Primer ID | Primer sequence (5′-3′) |
| | libUni-F-universal | AATGATACGGCGACCACCGAGATCTACACA |
| | | CACTCTTTCCCTACACGACGACGCTCTCTTCCGA |
| | | CAAGCAGAAGACGGCATACGAGATCGAGT |
| | libUni-D701-R | AATGTGACTGGAGTTCAGACGTGTGCTCTT |
| | | CAAGCAGAAGACGGCATACGAGATTCTCC |
| | libUni-D702-R | GGAGTGACTGGAGTTCAGACGTGTGCTCTT |
| | | CAAGCAGAAGACGGCATACGAGATAATGA |
| | libUni-D703-R | GCGGTGACTGGAGTTCAGACGTGTGCTCTT |
| | | CAAGCAGAAGACGGCATACGAGATGGAAT |
| | libUni-D704-R | CTCGTGACTGGAGTTCAGACGTGTGCTCTT |
| | | CAAGCAGAAGACGGCATACGAGATTTCTG |
| | libUni-D705-R | AATGTGACTGGAGTTCAGACGTGTGCTCTT |
| | | CAAGCAGAAGACGGCATACGAGATACGAA |
| | libUni-D706-R | TTCGTGACTGGAGTTCAGACGTGTGCTCTT |
| | | CAAGCAGAAGACGGCATACGAGATAGCTT |
| | libUni-D707-R | CAGGTGACTGGAGTTCAGACGTGTGCTCTT |
| | | CAAGCAGAAGACGGCATACGAGATGCGC |
| | libUni-D708-R | ATTAGTGACTGGAGTTCAGACGTGTGCTCTT |
| | | CAAGCAGAAGACGGCATACGAGATCATA |
| | libUni-D709-R | GCCGGTGACTGGAGTTCAGACGTGTGCTCTT |
| | | CAAGCAGAAGACGGCATACGAGATTTCG |
| | libUni-D710-R | CGGAGTGACTGGAGTTCAGACGTGTGCTCTT |
| | | CAAGCAGAAGACGGCATACGAGATGCGC |
| | libUni-D711-R | GAGAGTGACTGGAGTTCAGACGTGTGCTCTT |
| | | CAAGCAGAAGACGGCATACGAGATCTAT |
| | libUni-D712-R | CGCTGTGACTGGAGTTCAGACGTGT |
| | | GCTCTT |

2nd round PCR, take Ing of purified DNAfrom 1st round PCR as template, 10cycles, 15 μpl
system, anealed @ 65° C., using PrimeSTAR® Max DNA Polymerase (TaKaRa)
b. Characteristic sequence for read counting by R program

| Target | Feature | characteristic sequence for counting |
|---|---|---|
| HOXB13 target 6 | Wild Type | ttactttggagg |
| HOXB13 target 16 | Wild Type | tccgggaaccta |
| DYRKIA target 1 | Wild Type | tcagatggctgg |
| EMX1 target 1 | Wild Type | cttcccatcagg |
| EMX1 target 3 | Wild Type | aacccacgaggg |
| EMX1 target 4 | Wild Type | gcttgctgctgg |
| EMX1 target 5 | Wild Type | tgactagggtgg |
| EMX1 target 6 | Wild Type | gaagaagaaggg |
| EMX1 target 5 | HindIII | actAAGCTTggg |
| HOXB13 target 8 | C8 | gcggccagggtggctgcctG |
| HOXB13 target 8 | T8 | gcggccagggtggctgcctA |
| EMX1 target 6 | T5 | gaggaggaagggcctgagtT |
| EMX1 target 6 | C5 | gaggaggaagggcctgagtC |
| EMX1 target 6 | T6 | Tgagcagaagaagaagggct |
| EMX1 target 6 | C6 | Cgagcagaagaagaagggct |
| EMX1 target 6 | C5C6 | aggaggaagggcctgagtCC |
| EMX1 target 6 | T5T6 | aggaggaagggcctgagtTT |
| EMX1 target 6 | C5T6 | aggaggaagggcctgagtCT |
| EMX1 target 6 | T5C6 | aggaggaagggcctgagtTC |

c. R Program for read counting
library(ShortRead)
reads = readFastq(″libraryName″)
reads
total_counts = length(reads)
totalcounts
sequences = sread(reads)
dict = DNAStringSet(substr(sequences, 1,150))
hits = vcountPattern(″Wild Type characteristic sequence″, diet,max.mismatch = 0,
with.indels = FALSE)
wild_type_counts = sum(hits)
wildtypecounts
library(ShortRead)
reads = readFastq(″libraryName″)
reads
total_counts = length(reads)
totalcounts
sequences = sread(reads)
dict = DNAStringSet(substr(sequences, 1,150))
hits = vcountPattern(″expected_characteristic sequence″, diet,max.mismatch = 0,
with.indels = FALSE)
expected_sequence_counts = sum(hits)
expectedsequencecounts

Single cell cloning and genotyping. About 48 hours post transfection, single cells were isolated by limited dilution method and grown in 96-well plates. When reached confuent, cells were further propagated into 24-well plates and grew until confuent. Genomic DNA from single-cell clones was isolated with a TIANamp Genomic DNA Kit (TIANGEN, Cat #DP304-03) according to the manufacturer's instructions. The genotype of each clone was determined by getPCR assay and confirmed by Sanger sequencing of amplicon covering the cutting site. PCR amplifications were performed with high-fidelity PrimeSTAR® Max DNA Polymerase (TaKaRa, Cat #R045B) and primers as shown in Table 2a. PCR products were then subjected to Sanger sequencing (TsingKe Biological Technology or GeneWiz). To determine the exact sequence of each allele for heterozygous cells, the Sanger sequencing ab 1 files were directly analyzed with TIDE Web Tool (https://tide.nki.nl/). Alternatively, the amplicons were further cloned into vector and single cell clones were analyzed by Sanger sequencing.

Sensitivity of different DNA polymerases to mismatch. A variety of commercial DNA polymerase products were evaluated for their sensitivity to primer mismatch. They are Taq master mix (Vazyme, Cat #P111, Lot #511151), Premix TaqT™ (TaKaRa, Cat#RR901, Lot#A3001A), NOVA Taq-Plus PCR Forest Mix (Yugong Biolabs, Cat #EG15139, Lot#1393216101), DreamTaq Green PCR Master Mix (ThermoFisher, Cat #K1081, Lot#00291017), PlatinumTM Green Hot Start PCR Master Mix (Invitrogen, Cat #13001012, Lot#00401653), PrimeSTAR® Max DNA Polymerase (TaKaRa, Cat #R045, Lot#AI51995A), Phusion Hot Start II high-Fidelity PCR Master Mix (ThermoFisher, Cat#F-565, Lot#00633307) as well as Q5® Hot Start high-Fidelity DNA Polymerase (NEB, Cat#M0493). In a 20 μL reaction system, 10 ng of plasmid DNA was included as template and Thermal cycled with the programs as suggested by given product manuals. PCR products were then subjected to 2.0% agarose gel electrophoresis and Sanger sequencing directly. Gel images were acquired using Quantum-ST5 (VILBER LOURMAT, France) and analyzed with Quantum ST5 Xpress software.

Comparison of different qPCR SYBR green products in getPCR. To test the extensive usability of getPCR, multiple qPCR SYBR mix products were investigated including AceQ qPCR SYBR Green Master Mix (Vazyme, Cat #Q111-02), SYBRTM Select Master Mix (Applied Biosystems™, Cat #4472908), Power SYBR Green PCR Master Mix (Applied BiosystemsTM Cat #4367659), QuantiNova SYBR Green PCR Kit (QIAGEN, Cat #208054), FastStart Essential DNA Green Master (Roche, Cat#06402712001), NovoScript® SYBR One-Step qRT-PCR SuperMix (novoprotein, Cat #E092-01A), 2× T5 Fast qPCR Mix (TSINGKE, Cat #TSE202), UltraSYBR Mixture (CWBIO, Cat #CW0957), SYBR Premix Ex Taq (TaKaRa, Cat #RR420, A5405-1). Real-time qPCRs were run on the thermocyclers Rotor-Gene Q (Qiagen, Germany) or LightCycler® 96 Thermal cycler Instrument (Roche Applied Science, Germany). The PCR and qPCR conditions were set according to the manufacturer's protocol with given annealing temperature.

Statistical analysis. Student's t tests (two-tailed) were applied based on the results of Levene test to assess the statistical significance of getPCR results for single-cell clone genotyping using IBM SPSS Statistics version. The correlation between two different getPCR strategies were assessed with Pearson test using IBM SPSS Statistics version 21 software.

### Example 1. Watching Primer Design for getPCR

To make getPCR technique work, the principle for designing watching primer was determined in this example. Most indels occur surrounding the nuclease cutting site and small indels less than 15 bps accounts for the major part. In addition, to better distinguish indel sequences from wild-type sequences, this example focuses on the case of insertions or deletions with a small number of bases. In view of this, the inventors designed 26 plasmid constructs representing 1-15 bp indels to mimic in vivo nuclease induced genome editing targeting HOXB13 gene (FIG. 2a).

Two serials of primers with one to eight watching base(s) were designed (FIGS. 7a-c) and those with adequate amplification efficiency were chosen (FIG. 2b) for further examination of their ability in discriminating indels and wild-type DNA sequences. Theoretically, more watching base could increase the selectivity of watching primer. However, too many watching bases will make the mismatch move away from the 3′ end to the 5′ end and consequently impede the sensitivity of Taq polymerase on the contrary. When single direction watching primer employed, 3 to 5 watching bases exhibited preferable distinguish ability of indel sequences from wild type sequence for both reverse (FIG. 2c) and forward (FIG. 2d) primers. When forward and reverse watching primers applied in combination, 4 to 6 watching bases in sum could discriminate indels successfully (FIG. 2e, FIG. 7d). However, 5 or 6 additive watching bases showed higher background signal because of primer self-amplification (FIG. 2f, FIG. 7e). Therefore, 4 additive watching bases are ideal for designing combinational getPCR primers.

The 3′ end base of watching primer plays substantial roles in determining getPCR discrimination ability. The adenine base displayed best specificity and gave lowest non-specific amplification signal when mismatched with non-complementary bases. Cytosine came the second followed by guanine and thymine (FIG. 2g). When the mismatch located in the second last position, similar results were observed. The adenine base still displayed the best specificity and its mismatch with non-complementary bases was less tolerated by Taq polymerase (FIG. 2h). In addition, the 3′ end base type also determined the sensitivity of getPCR to mismatch happened upstream. Again, adenine base is the best choice and enables getPCR amplification most sensitive to mismatch happened at the second last position. It is worth noting that, if more than one mismatches occurred neighboring to the last base, the PCR amplification will be obviously destroyed whatever the last base is (FIG. 2i). Moreover, the closer to the 3′ end the mismatch is, the more sensitive to the mismatch the getPCR becomes (FIGS. 7f-g, FIGS. 8a-b).

To explore the potential mechanisms that enable getPCR sensitive to mismatch, this example compared the PCR amplification of 3′ end-mismatched primer with mismatch base-deleted primer. Interestingly, the deletion of mismatch base partially restored the amplification capacity in qPCR as well as common PCR analysis (FIGS. 7h-i, FIGS. 8a-b). Besides, high-fidelity DNA polymerases such as Phusion and Q5 that possess the proofreading 3′ to 5′ exonuclease activity could also restore the PCR amplification in part or completely. Sanger sequencing chromatograms of the PCR products showed that the mismatched nucleotide at the primer 3′ end was removed by the 3′ to 5′ exonuclease activity during polymerizing. On the contrary, Taq DNA polymerase without 3′ to 5′ exonuclease activity just tolerated and bypassed the mismatch directly (FIG. 8c). It indicates that, the mismatch impeded primer pairing with the template on one hand, and the spatial geometric hindrance caused by the mismatch further hampered Taq polymerase priming.

### Example 2. Parameters for Running a getPCR

The other issue needs to be addressed for getPCR is the optimum parameter, and this example focuses on annealing temperature in performing getPCR reaction. Along with the elevation of annealing temperature, the amplification specificity for matched wild template over mismatched indel templates obviously increased for all the four watching primers designed in example 1 (FIGS. 3a-d). However, when the annealing temperature increased to over 4° C. higher than Tm value, the PCR efficiency began to drop obviously on the contrary. Since optimal PCR efficiency is usually preferred for PCR amplification, the best selectivity of each watching primer was systematically evaluated under optimal PCR efficiency (FIGS. 3e-h). Intriguingly, no matter how many watching bases and total bases the primer had, the best selectivity was often observed at the annealing temperature about 4° C. higher than its Tm value (FIGS. 3e-h). With fixed watching base number, increasing primer Tm value by adding more bases at its 5′ end didn't dramatically alter the ability in discriminating indels. Three of the four types of primers exhibited steady ability in discriminating indels (FIGS. 3e-g). Only one type of primer showed slightly increased ability and reached optimum at Tm value around 65.8° C. (FIG. 3h). Therefore, in subsequent experiments watching primers were designed with Tm value around 65° C. and getPCR were performed with annealing temperature 69° C. for all kind of watching primers. More importantly, even though the increased annealing temperature over Tm value impeded the PCR efficiency, the basis of real-time PCR quantification, i.e., the linear correlation between the Ct value and logarithm template DNA quantity, was not affected at all for all the four types of primers (FIGS. 3i-l).

DNA polymerase plays essential roles in determining the discrimination ability of getPCR.

Even though varying in performance, almost all tested commercial Taq products in the example exhibited acceptable ability in discriminating indels from wild type sequence (FIG. 7j). However, when sensitivity to single-base mismatch was evaluated, seven from nine SYBR green qPCR products showed high applicable performance (FIGS. 8d-e).

Example 3. Research on the accuracy of quantitative genome editing by getPCR The ability of getPCR in quantifying genome editing efficiency was amplification evaluated with plasmids simulating genome editing indels as used in FIG. 2a. In the example, twenty-six plasmids with different indel mutations were combined equally and then mixed with wild construct at given ratio to mimic indel frequencies of 0%, 20%, 40%, 60%, 80% and 100%. The mixtures were subjected to indel frequency quantification by getPCR as well as the classic Surveyor method for comparison. When indel frequency is not higher than 20%, quantification results by Surveyor method could truly reflect the anticipated value. However, along with the further increasing of indel frequency, the observed value deviated from anticipated value progressively (FIGS. 4a, b). On the contrary, whether carrying 3, 4 or 5 watching bases, all the twelve getPCR strategies with different watching primers could accurately determine the indel frequencies (FIG. 4c, FIGS. 9a-c).

### Example 4. Application in Genotyping of Mimic Single-Cell Clones

The getPCR technique can also be used in single cell clone screening or offspring genotyping in genome editing experiments. Each indel construct as shown in FIG. 2a alone or equally combined with wild construct was used to mimic single-cell clone genomic DNA with double alleles or one allele modified respectively. All the three getPCR strategies could accurately determine the genotypes of all the clones. Not only determine if indels happened, but also clarify how many alleles carried indels accurately (FIGS. 4d-f). In addition, when any two getPCR strategies were analyzed in combination, their detection values exhibited extremely high correlation with a Pearson Correlation Coefficient equal to or higher than 0.995. Intriguingly, the combination of two getPCR strategies could dramatically improve the performance in defining the genotype (FIGS. 9d-f).

### Example 5. Determination of Editing Frequency and Genotype of Single-Cell Clones by getPCR

This example applied getPCR in the detection of genome editing with high-fidelity Cas9 variant and nine different gRNAs targeting HOXB13, DYRKIA or EMX1 gene in Lenti-X 293 T cells (FIG. 5b). The editing efficiency of each gRNA was determined by three different methods, including getPCR, NGS-based amplicon sequencing as well as Surveyor assay.

For all watching bases designed, the editing frequency determined by getPCR method was often comparable to the results from NGS method, which was believed to be the most reliable one. In contrast, the apparent editing frequency value determined by Surveyor method exhibited obvious deviation from the other two methods, especially at HOXB13 target 6 and target 16 where the editing efficiencies were high (FIG. 5a). The genome modified cells with gRNAs of HOXB13 target 6, EMX1 target 1 and 5 as well as DYRK1A target 1 were isolated single-cell colony and propagated in the example. The genomic DNA samples were prepared and subjected to genotyping by getPCR and verified through Sanger sequencing. Overall, all the single-cell clones from the genome editing experiments with these four gRNA targets were accurately genotyped by getPCR. Notably, not only the cell clones carrying indels could be detected, the one-allele modified cells and both-alleles modified cells could be successfully identified at the same time (FIGS. 5c-i, FIGS. 10a-b). For genome editing performed at HOXB13 gRNA target 6, 24 double allele-modified colonies and 5 single allele-modified colonies were accurately identified from total 42 colonies using two different designed getPCR primers containing 3 or 4 watching bases respectively (FIGS. 5c-d, FIG. 10h). Similarly, for editing at EMX1 target 5, 8 double allele-modified colonies and 5 single allele-modified colonies were identified by getPCR with primers carrying 4 watching bases, designed in forward or reverse direction. (FIGS. 5e-f, FIG. 10i). As to DYRK1A gRNA target 1, from total 53 colonies 11 were defined to be double allele-modified and 5 to be single allele-modified using getPCR with four different designed watching primers, which carrying 3, 4, or 5 watching bases in forward direction or 4 watching bases in reverse direction respectively (FIGS. 5g-h, FIGS. 10a-b,j). For EMX1 gene target 1, getPCR using the 4-watching base primer successfully identified 1 double allele-modified cell clone and 9 single allele-modified cell clones from 45 clones (FIG. 5i, FIG. 10k). Notably, any two differently designed getPCR exhibited highly correlated detection value and could help the genotyping when analyzed in combination (FIGS. 5j-1, FIGS. 10c-g).

### Example 6. Application of getPCR in HDR Detection in Cells and Genotype of Single-Cell Clones

The example illustrates the application of getPCR in the determination of repair efficiency of HDR-mediated genome editing (FIG. 6a). Genome editing experiments were performed in Lenti-X 293 T cells with Cas9 and EMX1 gRNA target 5 together with the HDR template designed to introduce a HindIII site neighbor to PAM sequence (FIG. 6b). The getPCR method as well as NGS-based amplicon sequencing and HindIII-mediated restriction fragment length polymorphism (RFLP) analysis were applied to determine the HDR efficiency. Two watching primers designed in forward and reverse direction respectively both could determine the HDR frequencies with comparable level to RFLP and NGS based methods (FIG. 6c). The HDR frequencies from three biological samples were evaluated to be around 25%. Furthermore, in genotyping of the 50 single-cell clones derived from this HDR experiment, both two watching primers successfully picked out all the 6 clones homozygous and all the 17 clones heterozygous for HDR event (FIGS. 6d-e). In addition, detection values with these two watching primers were highly consistent by a strong correlation (r=0.982, P=1.207×10⁻³⁶) and combination analysis could obviously promote the genotyping especially for heterozygous cell clones (FIG. 6f).

### Example 7. Determination of Base Editing Frequency and Genotype of Single-Cell Clones by getPCR

The example illustrates the application of getPCR in the detection of base editing frequency and genotype of single-cell clones by getPCR. The example applied getPCR in the base editing experiments with BE4 and gRNA of EMX1 target 6 or HOXB13 target 8 in Lenti-X 293 T cells (FIG. 6b). In quantification of base editing frequency, getPCR demonstrated comparable results to NGS-based amplicon sequencing method (FIGS. 6g-h). For EMX1 target 6, about 27% ‘C’ bases at the 5^{th} and 6^{th} positions of gRNA targeting sequence were converted into ‘T’. Intriguingly, the base editing at these two positions tended to happen simultaneously and generated T5T6 genotype (FIG. 6g). As to base editing with gRNA HOXB13 target 4, which was designed to terminate the open reading frame early by introducing an in-ahead stop codon ‘TAG’, the C-to-T editing efficiency at the 8^{th} position was around 15%, (FIG. 6h).

The Lenti-X 293 T cells that underwent base editing at EMX1 target 6 or HOXB13 target 8 were further isolated single-cell clones and subjected to genotyping with getPCR method. For base editing at EMX1 target 6, 25 out of 46 clones were determined to carry C-to-T conversion at the 5^{th} position (FIGS. 6j-k), and 22 out of 46 clones were proven to carry C-to-T conversion at the 6^{th} position by getPCR analysis (FIGS. 61-m). Clone E01, E29 and E70 might contain extra base other than C and T at 5^{th} position and clone E24 might carry such extra base at 6^{th} position as suggested by the missing percentage of base composition in getPCR detection results. Sanger sequencing of these clones showed that C-to-G base editing happened at the 5^{th} position of clone E01 and E29 and at the 6^{th} position of clone E24 (FIGS. 11a-c). Specifically, clone E70 didn't carry base conversion other than C to T at the 5^{th} nucleotide but had an A-to-T editing at the −8 nucleotide of gRNA targeting sequence on one allele (FIG. 11c). This A-to-T mutation can be mapped to the 14^{th} nucleotide of primer from the 3′ end and impeded the primer annealing to this allele, which in turn resulted in missed getPCR signal. HEK-293 cells, where Lenti-X 293 T came from, has been reported to be near triploid with 62-70 chromosomes per cell. Consistently, the allele percentages of heterozygous clones were usually found to be around 33% or 66% but not 50% in getPCR analysis (FIGS. 6j,l).

Furthermore, these triploid characteristics were further validated in Sanger sequencing analysis, where the heterozygous allele peak maps of the two heterozygous alleles typically had a two-fold rather than comparable interrelationship in height (FIG. 11c). For example, the percentages of T and C base at the 5^{th} nucleotide position of clone E11 were determined to be 28.8% and 62.9% respectively in getPCR analysis, and consistently the peak height of C base was nearly twice of T in Sanger sequencing. However, even though Sanger sequencing results available, ten clones were still unknown for allele specific genotype which were heterozygous at both 5^{th} and 6^{th} nucleotide (FIG. 11c). Four watching primers were designed in the example to further genotyping these clones through getPCR method (FIG. 6b), and the exact allele-specific genotypes of these clones were successfully determined (FIG. 6i). Clone E02 and E15 were defined to be C5C6/C5C6/T5T6, and clone E33, E39, E40 as well as E49 were proven to be C5C6/T5T6/T5T6. Clone E01 and E29 were found both to be C5C6/T5T6/G5C6 and clone E24, E34 were finally determined to be C5C6/T5C6/T5G6 and C5C6/T5T6/T5C6 respectively.

For base editing at HOXB13 target 8 to introduce an in-frame stop codon, 14 out of 49 clones in the example were determined to carry C-to-T conversion at the 8^{th} position, which would have resulted in an early stop codon (FIGS. 6n-o). Notably clone 35 could possibly carry extra base other than C and T bases at this position as suggested by missing part in base percentage in getPCR detection. Sanger sequencing chromatograms showed that a C-to-G base editing happened at the 8th position on one of the three alleles (FIG. 12a-b). Similarly, getPCR could also determine the delicate genotypes of heterozygous clones as verified by sanger sequencing. For example, six clones S15, S47, S44, S18, SO2 and S35 were genotyped to be C/C/T at the 8^{th} nucleotide of HOXB13 gRNA target 4 sequence.

The foregoing descriptions are only preferred embodiments of the application and are not intended to limit the application. Although the application has been described in detail with reference to the foregoing embodiments, for those skilled in the art, modifications to technical solutions recorded in the foregoing embodiments or equivalent replacement of some of the technical features may still be made. Any modification, equivalent replacement, or improvement made within the spirit and principle of the present application shall fall within the protection scope of the present application.

1. A method for detecting the frequency of nuclease-induced indel occurrence, wherein comprising: adding primers and Taq DNA polymerase to a genomic DNA sample to be tested, amplifying wild-type DNA in the genomic DNA sample, and quantifying the proportion of wild-type DNA by PCR, thereby confirming the frequency of indel occurrence in the genome; the primer is sequence-matched to the wild-type DNA and the sequence of the primer covers the nuclease cutting site.

2. The method according to claim 1, wherein nucleases comprises Cas9 nucleases, zinc finger nucleases, transcription activator-like effector nucleases, CRISPR RNA guide Fokl nucleases, and paired cas9 nickase; further, the nucleases are Cas9 nucleases; the primers is designed to span Cas9 nuclease cutting site near its 3′ end.

3. The method according to claim 2, wherein the primer comprises a watching sequence, the watching sequence is a sequence between the nuclease cutting site and the 3′ end of the primer, having a length of 1 to 8; or the primer is a pair of nucleotide sequences designed in forward and reverse direction, and the length of the watching primer base is 4 bp.

4. The method according to claim 3, wherein the 3′ end base of watching primer is an adenine base or a cytosine or a guanine base.

5. The method according to claim 1, wherein an annealing temperature on amplification is Tm˜Tm+4° C.

6. A kit for detecting the frequency of nuclease-induced indel occurrence, comprising primers, Taq DNA polymerase and PCR detection reagents.

7. Application of the kit according to claim 6 in evaluating genome editing efficiency and/or single-cell clone screening.

8. A method for genotyping of single-cell clones, wherein comprising: using wild-type DNA in genome to be tested as a template, designing primers against alleles, extracting genomic DNA of single-cell clones to be tested, and detecting whether the alleles in the genomic DNA of single-cell clones have indels by the method of claim 1 thereby achieving single-cell colony genotyping.

9. A method for detecting HDR efficiency, wherein comprising: designing primers for the genomic DNA repaired by HDR in the genome to be detected, extracting the genomic DNA to be detected, and detecting the occurrence probability of HDR by adopting the method of claim 1; the percentage of DNA repaired by HDR is the HDR efficiency.

10. A method for detecting the editing efficiency of a base editor, wherein comprising: taking the genome DNA to be detected as a template, designing primers for a target sequence after base editing, and adopting the method of claim 1 to detect the occurrence probability of base editing in the genome, which is the editing efficiency of the base editor.
